(19) United States
(12) Patent Application Publication
(10) Pub. No.: US 2026/0364160 A1
(43) Pub. Date: Nov. 19, 2026

(54) EPSILON TOXIN FROM CLOSTRIDIUM PERFRINGENS AS A VACCINE

(71) Applicant: One Health Ventures Limited, London (GB)

(72) Inventors: Richard William TITBALL, Devon (GB); Monika BOKORI-BROWN, Exeter (GB); Helen MORCRETTE, Exeter (GB); Nicholas Peter LEWIS, Berkshire (GB)

(21) Appl. No.: 19/224,517

(22) Filed: Aug. 7, 2025

Related U.S. Application Data
(63) Continuation of application No. 18/915,091, filed on Oct. 14, 2024.
Application No. 18/915,091 is a continuation of application No. 16/976,869, filed on Aug. 31, 2020, now Pat. No. 12,144,854.

(51) Int. Cl. A61K 39/08; A61K 39/00; A61P 37/06; C07K 14/33; C07K 16/1282

(52) U.S. Cl. CPC A61K 39/08; A61P 37/06; C07K 14/33; C07K 16/1282; A61K 2039/552

(57) ABSTRACT

This invention relates to methods and compositions for detecting, diagnosing, preventing, treating or ameliorating the symptoms of a demyelinating condition selected from:: enterotoxemia (ET), multiple sclerosis (MS), clinically definite MS (CDMS), clinically isolated syndrome (CIS), neuromyelitis optica spectrum disorder (NMOSD), optic neuritis (ON), neuromyelitis optica (NMO), myelitis, myelitis, transverse myelitis (TM), a disease or condition characterised by the increase or presence of antibodies against aquaporin-4 (AQP-4) and/or astrocyte damage, and acute disseminated encephalomyelitis (ADEM) in a human or animal subject in need. The methods comprise administering to the subject a composition comprising an effective amount of an agent that directly or indirectly interferes with epsilon toxin (ETX) produced by Clostridium perfringens type B or type D bacterial strain, an ETX-binding receptor, or an interaction of ETX with its binding receptor so as to inhibit or suppress ETX modulated receptor signalling activities. The invention also provides novel polypeptides useful as a vaccine against diseases caused by or associated with the epsilon toxin of Clostridium perfringens.

### FIELD OF THE INVENTION

This invention relates to methods and compositions for detecting, diagnosing, preventing, treating or ameliorating the symptoms of a demyelinating condition.

### BACKGROUND

The rod-shaped, spore-forming, Gram-negative, anaerobe bacterium Clostridium perfringens is able to produce at least 17 toxins, making C. perfringens one of the most pathogenic species in the Clostridium genus. Depending on its ability to produce the four typing toxins, namely α-, β-, ε-, and i-toxin, C. perfringens strains are classified into one of five toxinotypes, referred to as types A-E (Petit et al. (1999) Trends Microbiol. vol. 7, 104-110).

Epsilon toxin (Etx) is produced by toxinotypes B and D. These strains are responsible for a severe disease called enterotoxemia, which affects predominantly sheep and lambs but also causes infections in other ruminant species, including goats and calves (Songer (1996) Clin. Microbiol. Rev. vol. 9, 216-234). Enterotoxemia in naturally infected animals is usually characterised by systemic lesions in sheep and enterocolitis in goats. In addition to the typing toxins, the bacterium is able to produce a variety of so-called minor toxins such as β1, β2, δ, θ, λ, μ, v, and enterotoxin (Rood (1998) Annu. Rev. Microbiol. vol. 52, 333-360).

The most important factor in initiating disease in sheep and other ruminants is overeating rich food, resulting in the presence of high amounts of carbohydrates in the intestine. This leads to disruption of the microbial balance in the gut, leading to proliferation of C. perfringens and consequent overproduction of Etx. The toxin causes an increase in intestinal permeability, facilitating its entry into the bloodstream and allowing its dissemination to the main target organs of the kidneys and the brain (McDonel (1980) Pharmacol Ther 10(3): 617-655). Here, intoxication results in fluid accumulation due to increased permeability of blood vessels. Accumulation in the central nervous system results in neurological disorder rapidly leading to death (Finnie (2003) Aust. Vet. J. vol. 81, 219-221).

More recently, Etx has been suggested to play a role in the development of multiple sclerosis in humans (Rumah et al. (2013) PLoS One 8: e76359).

Multiple sclerosis (MS) is a demyelinating disease in which the insulating covers of nerve cells in the brain and spinal cord are damaged. This damage disrupts the ability of parts of the nervous system to communicate, resulting in a range of symptoms, which can include double vision, blindness in one eye, muscle weakness, trouble with sensation, or trouble with coordination. The condition often begins as a clinically isolated syndrome (CIS) over a period of time, before being confirmed as Clinically Definite MS (CDMS).

Neuromyelitis optica spectrum disorder (NMOSD) is a rare neurological condition characterised by episodes of optic neuritis (ON), transverse myelitis (TM), together with one or more other diagnostic criteria including in some cases the presence of a specific antibody, aquaporin-4 (AQP-4).

Optic neuritis (ON) is a demyelinating inflammation of the optic nerve. It is frequently associated with multiple sclerosis. The autoimmune disease neuromyelitis optica (NMO) is a heterogeneous condition consisting of the simultaneous inflammation and demyelination of the optic nerve (optic neuritis) and the spinal cord (myelitis). Approximately 80% of patients diagnosed with NMO test positive for Aquaporin 4 (AQP-4) antibodies (http://www.nmouk.nhs.uk/healthcare-professionals/aqp4-antibodies). Zamvil et al., (Neurotherapeutics (2018) 15: 92-101) speculate that gut microbiota, and possibly C. perfringens itself, could participate in NMO pathogenesis.

Transverse myelitis (TM) is an inflammation of both sides of one section of the spinal cord, and may also result in myelin damage.

Acute disseminated encephalomyelitis (ADEM) is characterized by a brief but widespread attack of inflammation in the brain and spinal cord that damages myelin. ADEM often follows viral or bacterial infections, or less often, vaccination for measles, mumps, or rubella. ADEM typically damages white matter, leading to neurological symptoms such as visual loss (due to inflammation of the optic nerve) in one or both eyes.

Etx is expressed with a signal sequence that directs export of the prototoxin from the bacterium (McDonel (1986) in Pharmacology of bacterial toxins eds. Dorner & Drew, Pergamon Press, 477-517). In development of disease, the relatively inactive prototoxin is converted to the active toxin by proteolytic cleavage in the gut lumen, either by digestive proteases of the host, such as trypsin and chymotrypsin (Bhown & Habeerb (1977) Biochem. Biophys. Res. Commun. vol. 78, 889-896), or by C. perfringens λ-protease (Minami et al. (1997) Microbiol. Immun. vol. 41, 527-535). Proteolytic activation of Etx can also be achieved in vitro by controlled proteolysis (Hunter et al. (1992) Infect. Immun. vol. 60, 102-110). Depending on the protease, proteolytic cleavage results in the removal of 10-13 amino-terminal and 22-29 carboxy-terminal amino acids (Bhown & Habeerb (1977); Minami et al. (1997)). Maximal activation occurs when both N- and C-termini are cleaved (Worthington & Mulders (1977) Infect. Immun. vol. 18, 549-551).

The 3D structure of Etx has been determined (Cole et al. (2004) Nature Structural & Molecular Biology vol. 11, 797-798) and reveals a molecule composed mainly of β-sheets, which can be divided into three functional domains. Domain I at the N-terminus contains the suggested receptor interaction region. Domain II in the middle contains an amphipathic β-hairpin, which is predicted to play a role in membrane insertion. Domain III at the C-terminus contains the C-terminal peptide, which has to be removed for activation to occur.

Epsilon toxin is an aerolysin-like β-pore forming toxin (β-PFT), with the amphipathic β-hairpin loops inserting into the membrane to form β-barrel structures. The overall fold of Etx shows similarity to the structure of aerolysin from the Gram-negative bacterium Aeromonas hydrophila (Parler et al. (1994) Nature vol. 367, 292-295), to parasporin-2 (PS) from Bacillus thuringiensis (Akiba et al. (2009) J. Mol. Biol. vol. 386, 121-133) and to a pore-forming lectin, LSL, from Laetiporus sulphurous (Mancheno et al. (2005) J. Biol. Chem. vol. 280, 17251-17259). The structural similarities between these toxins are most striking in their two C-terminal domains. Their N-terminal domains show a greater structural variation, which is likely to account for their differences in target cell specificities and potencies (Bokori-Brown et al. (2011) FEBS J. vol. 278, 4589-4601).

In aerolysin, the two amino-terminal domains (Domains I-II) are thought to play a role in binding to cell surfaces with overlapping functions (MacKenzie et al. (1999) J. Biol. Chem. vol, 274, 22604-22609) and it has been suggested that domain I of Etx, which is equivalent to domain II of aerolysin, performs a similar function (Cole et aL. (2004)), but this has yet to be demonstrated. Domain II of aerolysin contains the mannose 6-phosphate binding loops. However, the residues of domain II involved in mannose-6-phosphate binding in aerolysin are not conserved in domain I of Etx, suggesting that the structural variation in the N-terminal receptor binding domains of these toxins is likely to account for the differences between their target cell specificities.

Etx is unique among β-PFTs because it is highly potent and has high cell specificity. Because of its high potency, Etx is considered to be a potential biological weapon for international terrorism by the U.S. Government Centres for Disease Control and Prevention (Morbidity and Mortality Weekly Report (MMWR) Recommendations and Reports (2000) vol. 49, 1-14). The 50% lethal dose (LD₅₀) of Etx in mice after intravenal injection is typically 100 ng/kg (Gill (1982) Microbiol. Rev. vol. 46, 86-94), making Etx the most potent clostridial toxin after botulinum neurotoxin. Etx also shows high cell specificity. Among the many cell lines tested, only four have been identified to be susceptible to the toxin. These include kidney cell lines of dog (MDCK (Knight et al. (1990) Biologicals vol. 18, 263-270)), mouse (mpkCCDcl4 (Chassin et al. (2007) Am. J. Physiol. Renal Physiol. vol. 293, F927-937)) and human (G-402 (Shortt et al. (2000) Hum. Exp. Toxicol. vol. 19, 108-116) and ACHN (Ivie et al. (2011) PloS ONE vol. 6, e17787) origin. Most in vitro studies on Etx have been carried out using the Madin-Darby Canine Kidney (MDCK) cell line, as this cell line is the most susceptible to the toxin (Payne et al. (1994) FEMS Microbiol. Lett. vol. 116, 161-167). The dose of Etx to kill 50% of MDCK cells (CT₅₀) is reported to be as low as 15 ng/ml.

The binding of Etx to MDCK cells is associated with the formation of a stable, high molecular weight complex (Petit et al. (1997) J. Bacteriol. vol. 179, 6480-6487). Intoxicated cells undergo morphological changes that include swelling and membrane blebbing before cell death (Petit et al. (1997) J. Bacteriol. vol. 179, 6480-6487). The rapid toxin-induced cell death and the specificity of epsilon toxin for only a few cell lines suggest the presence of a specific receptor(s) on target cells. Etx acts by binding to host cells and there is evidence that seven monomers of the protein assemble into a pore which spans the cell membrane (Miyata et al. (2002) J Biol Chem. 277: 39463-8.), resulting in unregulated ion movement across the membrane and cell death. Toxicity appears to be a consequence of the formation of pores in the target cell membrane (Petit et al. (2001) J. Biol. Chem. vol. 276, 15736-15740).

The identity of the cell surface receptor for the toxin is still not fully clarified. There is evidence that the toxin binds to the hepatitis A virus cell receptor 1 proteins (HAVCR1) on MDCK.2 cells (Ivie et al. (2011) PLoS One 6: e17787). More recently, evidence has been presented that the receptor is myelin and lymphocyte protein (MAL) (Rumah et al. (2015) PLoS Pathog. 11: e1004896). CHO cells which are normally highly resistant to the toxin become sensitive when expressing MAL, and MAL knock-out mice are reported to be highly resistant to the toxin (Rumah et al. (2015)).

A number of commercial vaccines are available for the prevention of enterotoxaemia. These vaccines are typically produced by treating a C. perfringens culture filtrate with formaldehyde, resulting in detoxification of Etx. These vaccines contain a wide range of proteins in addition to Etx, and there can be considerable batch to batch variation in the immunogenicity of these preparations. Inflammatory responses following vaccination have been reported to result in reduced feed consumption. These shortfalls have prompted work to devise improved vaccines, and a number of recombinant immunogens have been reported including formaldehyde treated Etx produced from E. coli (Lobato et al. (2010) Vaccine 28:6125-7) and site directed mutants (genetic toxoids) of Etx with reduced toxicity (Kang et al. (2017) Human vaccines & immunotherapeutics 13:1598-608). Site directed mutants overcome the problem of batch to batch variation in immunogenicity associated with chemical detoxification methods of vaccine production. However, the high potency of the toxin can make it difficult to abolish toxicity. The toxicity of the mutants has been assessed using either MDCK cell cultures (Ivie and McClain (2012) Biochemistry 51:7588-95; Kang et al. (2017)) or in mice.

A site-directed mutant of Etx, Y30A-Y196A, was reported to have >430-fold decrease in cytotoxicity towards MDCK.2 cells compared with the wild type toxin and showed reduced but not abolished toxicity in mice (Bokori-Brown et al., (2014) Vaccine vol. 32, 2682-2687).

There is a continued need to identify improved molecules with potential for use as a vaccine against disease caused by or associated with the presence of Etx and/or caused by infection by C. perfringens.

### DETAILED DESCRIPTION OF THE INVENTION

This invention relates to methods and compositions for detecting, diagnosing, preventing, treating or ameliorating the symptoms of a demyelinating condition selected from: enterotoxemia (ET), multiple sclerosis (MS), clinically definite MS (CDMS), clinically isolated syndrome (CIS), neuromyelitis optica spectrum disorder (NMOSD), optic neuritis (ON), neuromyelitis optica (NMO), myelitis, transverse myelitis (TM), a disease or condition characterised by the increase or presence of antibodies against aquaporin-4 (AQP4) and/or astrocyte damage, and acute disseminated encephalomyelitis (ADEM) in a human or animal subject in need. The methods comprise administering to the subject a composition comprising an effective amount of an agent that directly or indirectly interferes with epsilon toxin (Etx) produced by Clostridium perfringens type B or type D bacterial strain, an Etx-binding receptor, or an interaction of Etx with its binding receptor so as to inhibit or suppress Etx modulated receptor signalling activities.

The invention relates to novel polypeptides useful as a vaccine against diseases caused by or associated with the epsilon toxin of Clostridium perfringens, particularly in animals susceptible to development of enterotoxemia and in the treatment of a demyelinating condition.

The present inventors have found that subjects having demyelinating conditions, such as enetrotoxemia (ET), neuromyelitis optica (NMO) and transverse myelitis (TM), the latter two being examples of neuromyelitis optica spectrum disorder (NMOSD), also tested positive for the presence of epsilon toxin (Etx) produced by Clostridium perfringens type B or type D and/or tested positive for the presence of antibodies against aquaporin-4 (AQP4).

Demyelinating conditions are characterised by damage to the myelin sheath and include conditions selected from: enterotoxemia (ET), multiple sclerosis (MS), clinically definite MS (CDMS), clinically isolated syndrome (CIS), neuromyelitis optica spectrum disorder (NMOSD), optic neuritis (ON), neuromyelitis optica (NMO), myelitis, transverse myelitis (TM), a disease or condition characterised by the increase or presence of antibodies against aquaporin-4 (AQP4) and/or astrocyte damage, and acute disseminated encephalomyelitis (ADEM).

According to a first aspect of the present invention, there is provided a method for preventing, treating or ameliorating the symptoms of a demyelinating condition selected from: enterotoxemia (ET), multiple sclerosis (MS), clinically definite MS (CDMS), clinically isolated syndrome (CIS), neuromyelitis optica spectrum disorder (NMOSD), optic neuritis (ON), neuromyelitis optica (NMO), myelitis, transverse myelitis (TM), a disease or condition characterised by the increase or presence of antibodies against aquaporin-4 (AQP-4) and/or astrocyte damage, and acute disseminated encephalomyelitis (ADEM) in a human or animal subject in need. The methods comprise administering to the subject a composition comprising an effective amount of an agent that directly or indirectly interferes with epsilon toxin (ETX) produced by Clostridium perfringens type B or type D bacterial strain, an ETX-binding receptor, or an interaction of ETX with its binding receptor so as to inhibit or suppress ETX modulated receptor signalling activities.

According to a second aspect of the invention, the composition comprising an agent that directly or indirectly interferes with epsilon toxin (ETX) produced by Clostridium perfringens type B or type D bacterial strain, an ETX-binding receptor, or an interaction of ETX with its binding receptor so as to inhibit or suppress ETX modulated receptor signalling activities is a C. perfringens epsilon toxin (Etx) polypeptide having reduced toxicity to cells expressing Myelin And Lymphocyte (MAL) protein and comprising a modified domain III compared to wild type Etx polypeptide having sequence SEQ ID NO:65, wherein said reduced toxicity is relative to SEQ ID NO: 65 and/or SEQ ID NO: 14 and wherein said Etx polypeptide is capable of binding to at least one antibody which binds to a sequence represented by SEQ ID NO:65 and/or SEQ ID NO:14.

Alternatively or additionally to the ability to bind an antibody which binds to SEQ ID NO:13 and/or SEQ ID NO:14, the polypeptide may bind to at least one antibody which binds to SEQ ID NO:11, the non-activated prototoxin form of the epsilon toxin.

A modified domain III may be any modification in the glycan (β-octyl-glucoside) binding site of domain III and/or in the sugar binding capacity of domain III and/or a modification of domain III which confers to the Etx polypeptide a reduced capacity to bind to CHO cells expressing MAL, compared to the corresponding wild type sequence when activated.

A modified domain III may comprise one or more amino acid mutations within the amino acid sequences making up domain III (SEQ ID NOs 1, 2 and 3): VYVGKALLTNDTQQEQKLKSQSFTCK (SEQ ID NO: 1), THNVPSQDILVPANTTVEVIAYLK (SEQ ID NO: 2); and DELIVKVRNLNTNNVQEYVIPVDKKEKSNDSNIVKYRSLYIKAPGIK (SEQ ID NO: 3), a mutation being a substitution or deletion of at least 1, 2, 3, 4, 5, 6, 7, 8, 9, 10 or more or substantially all of the amino acid residues in domain III (SEQ ID NOs 1, 2 and 3). Where there is more than one mutation in domain III, the mutations may optionally be a combination of substitutions and deletions.

The modified domain III may comprise one or more of the following mutations in SEQ ID NOs 1 and 2: VYVGKALLTNDTQQEQKLKSQSFTCK (SEQ ID NO: 1), where the underlined and bold V and F may be substituted for any other amino acid or may be deleted. For example, V may be substituted for F (V[F]) and/or F substituted for A (F[A]); THNVPSQDILVPANTTVEVIAYLK (SEQ ID NO: 2), where the underlined and bold H, V and A may be substituted for any other amino acid or may be deleted. For example, H may be substituted for A (H[A]), V substituted for A (V[A]) and/or A substituted for F (A[F]).

The polypeptide of the invention may comprise a mutation at a position as set out in Table 1 below, which gives examples of suitable mutations in domain III (SEQ ID NOs 6-10). The mutations shown in SEQ ID NOs 4 and 5 are domain I mutations. The Etx polypeptide of the invention may therefore comprise one or more of the following sequences representing a mutation in domain III: SEQ ID NO: 6, SEQ ID NO: 7, SEQ ID NO: 8, SEQ ID NO: 9 and SEQ ID NO: 10; or one or more of the mutations comprised in SEQ ID NOs 1 and/or 2.

TABLE 1
MUTATIONS COMPRISED IN SEQ ID NOS 4-10

| | | | Mutation position |
|---|---|---|---|
| | | | compared to |
| | | | equivalent in |
| SEQ ID NO | Sequence | Change | SEQ ID NO: 65 |
| SEQ ID NO: 4 | RMEKYXPNAM | Where X is any amino acid | 30 |
| | | other than Y (tyrosine), | |
| | | preferably wherein X is A | |
| | | (alanine) or wherein X is a | |
| | | deletion | |
| SEQ ID NO: 5 | GEIPSXLAFP | Where X is any amino acid | 196 |
| | | other than Y (tyrosine), | |
| | | preferably wherein X is A | |
| | | (alanine) or wherein X is a | |
| | | deletion | |
| SEQ ID NO: 6 | SKEITXNVPS | Where X is any amino acid | 149 |
| | | other than H (histidine), | |
| | | preferably wherein X is A | |
| | | (alanine) or wherein X is a | |
| | | deletion | |
| SEQ ID NO: 7 | LEDVYXGKAL | Where X is any amino acid | 72 |
| | | other than V (valine), | |
| | | preferably wherein X is F | |
| | | (phenylalanine) or wherein X | |
| | | is a deletion | |
| SEQ ID NO: 8 | LKSQSXTCKN | Where X is any amino acid | 92 |
| | | other than F (phenylalanine), | |
| | | preferably wherein X is A | |
| | | (alanine) or wherein X is a | |
| | | deletion | |
| SEQ ID NO: 9 | NTTVEXIAYL | Where X is any amino acid | 166 |
| | | other than V (valine), | |
| | | preferably wherein X is A | |
| | | (alanine) or wherein X is a | |
| | | deletion | |
| SEQ ID NO: 10 | TVEVIXYLKK | Where X is any amino acid | 168 |
| | | other than A (alanine), | |
| | | preferably wherein X is F | |
| | | (phenylalanine) or wherein X | |
| | | is a deletion | |

The mutation comprised within any one or more of SEQ ID NOs 4-10 may be comprised within a stretch of at least 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 255, 260, 261, 262, 263 contiguous amino acids from SEQ ID NO: 13, comprising one or more of SEQ ID NOs 4-10.

The invention need not be limited to the specific mutations of Table 1 and any mutation(s) which affect the sugar binding capacity of domain III and/or which mutation(s) confer a reduced capacity of an Etx polypeptide to bind to (CHO) cells expressing MAL equally form part of the present invention. A person skilled in the art could readily identify suitable mutations using known tools and routine techniques, for example those described herein.

As shown in Table 2 below, SEQ ID NO: 11 is full length wild type C. perfringens epsilon toxin and SEQ ID NO: 12 is the same sequence, but lacking the first 32 amino acids. This sequence, SEQ ID NO:12, is the sequence published for the crystal structure (see the Research Collaboratory for Structural Bioinformatics (RCSB) databank at www.rcsb.org/pdb; PDB ID: 1UYJ).

SEQ ID NO:65 is the trypsin activated wild type C. perfringens epsilon toxin which remains after trypsin protease cleavage, with N- and C-termini removed. SEQ ID NOs:11 and 65 have 79% identity at the global alignment level, when determined as outlined below.

SEQ ID NO:13 is a recombinant toxin comprising SEQ ID NO:65 and two additional amino acid residues at the N-terminal end. SEQ ID NO:14 is a sequence equivalent to SEQ ID NO:13 but with a H>A mutation at position 151 of SEQ ID NO:13 (mentioned in the Examples below as the H149A mutation, with the difference in residue numbering explained below). This is a variant of the activated toxin which may be studied in the laboratory at ACGM level 2 (Oyston et al. (1998) Microbiol. vol. 144 (Pt 2), 333-341) and so may be more convenient practically for determining antibody binding. Inclusion of the H149A mutation described herein would doubly ensure that the polypeptide according to the invention could be used at ACGM level 2.

TABLE 2
IDENTITY OF SEQUENCES

| SEQ | |
|---|---|
| ID | |
| NO | Identity of sequence |
| 11 | full length wild-type native epsilon toxin |
| 12 | sequence used to obtain crystal structure |
| | (PDB ID:1YUJ) |
| 13 | trypsin activated recombinant epsilon toxin |
| 14 | trypsin activated recombinant epsilon toxin |
| | with H149A mutation |
| 15 | recombinant epsilon toxin sequence |
| 65 | trypsin activated wild-type epsilon toxin |

Reference herein to the following mutation positions mentioned in Table 1, i.e. 30, 196, 72, 92, 149, 166 and 168 are as counted from position 1 of SEQ ID NO: 65. The same residue positions may be found in SEQ ID NO: 11 (counting starting from residue 46, i.e. SEQ ID NO:65 lacks residues 1-32 of SEQ ID NO:11 (the signal sequence) and residues 33-45 (the N-terminal pro-peptide)). The same residue positions may be found in SEQ ID NO: 12 (counting staring from residue 14, i.e. SEQ ID NO:65 lack residues 1-13 of SEQ ID NO:12 (the N-terminal pro-peptide)). The same residue positions may be found in SEQ ID NOs 13 and 14 (counting starting from residue 3, i.e. SEQ ID NO:65 lacks residues 1-2 of SEQ ID NOs: 13 and 14 (part of a synthetic signal sequence)). The same residue positions may be found in SEQ ID NO: 15 (counting starting from residue 25, i.e. SEQ ID NO:65 lacks residues 1-24 of SEQ ID NO:15 (synthetic signal sequence). A person skilled in the art would readily be able to determine mutations at positions equivalent to positions 30, 196, 72, 92, 149, 166 and 168 in any given Etx polypeptide.

Reference to “Y30A-Y196A” and “Y43A-Y209A” double mutants are used interchangeably herein to refer to the same mutation positions within the Etx wild-type sequence (represented by SEQ ID NO:65), with the position numbering depending on whether the position is within activated toxin or the inactive precursor thereof. The positions 30 and 196 when the double mutant is referred to as Y30A-Y196A are counted from the start of the activated protein (position 1 of the sequence shown in SEQ ID NO:65) and the equivalent positions 43 and 209 when the double mutant is referred to as Y43A-Y209A are counted from the start of the inactive precursor (i.e., starting from position −13 of the sequence shown in FIG. 2 or starting from position 1 of SEQ ID NO: 12). The mutation positions for the V72F, F92A, H149A, V166A, A168F mutations are as found in SEQ ID NO:65.

In a previous study (WO 2013/144636), it was shown that Y30A-Y196A double mutants (located in domain I) markedly reduced the ability of the toxin to bind to and kill MDCK cells and had reduced toxicity in mice, suggesting that Y30A-Y196A mutant could form the basis of an improved recombinant vaccine against enterotoxemia.

The previous study however used the MDCK cell line to measure cytotoxicity. Surprisingly, the double mutant did not give the same reduced toxicity results in CHO cells expressing MAL. It has surprisingly now been found that modifying domain III of the Etx polypeptide, for example by introducing one or more mutations into domain III, reduces toxicity in CHO cells expressing MAL. The modification to domain III may, for example, be any modification described herein.

The modification(s) to domain III therefore improved known vaccine candidate Y30A-Y196A as described in WO 2013/144636. Most in vitro studies on Etx have been carried out using the MDCK cell line, as this cell line was considered the most susceptible to the toxin (Payne et al. (1994) FEMS Microbiol. Lett. vol. 116, 161-167). Given that Etx vaccine candidates will likely not have been tested on (CHO) cells expressing MAL, the present invention provides an opportunity to improve existing Etx vaccines and Etx vaccine candidates by modifying domain III as described herein.

The polypeptide according to the invention may comprise a modified domain III compared to the wild type polypeptide SEQ ID NO:65, the polypeptide according to the invention showing reduced toxicity compared to an Etx polypeptide comprising SEQ ID NO: 4 and SEQ ID NO: 5 or compared to an Etx polypeptide comprising SEQ ID NO: 4, SEQ ID NO: 5 and SEQ ID NO: 6. In this context, the “Etx polypeptide” may be one having at least about 60%, 65%, 70%, 75%, 80%, 85%, 90%, 95%, 96%, 97%, 98%, 99% or about 100% sequence identity at a global level to SEQ ID NO:65, the polypeptide being capable of binding to at least one antibody which can bind to SEQ ID NO:65.

SEQ ID NOs 4 and 5 are present in the Y30A-Y196A double mutant, but the mutation of other tyrosine residues in domain I has also been shown to be effective in reducing toxicity of MDCK cells. For example, the mutated tyrosine residue(s) may, for example, be mutations (substitutions or deletions) of one or more of Y29, Y33, Y42, Y43, Y49 and/or Y209, the residue numbering here being counted from the start of the inactive precursor (i.e., starting from position −13 of the sequence shown in FIG. 2 or starting from position 1 of SEQ ID NO:12). The tyrosine mutation(s) may be comprised within a stretch of at least 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 255, 256, 257, 258, 259 or 260 contiguous amino acids from SEQ ID NO:65.

According to the present invention, there is also provided an Etx polypeptide having a modified domain III compared to wild type polypeptide SEQ ID NO:65 and showing reduced toxicity compared to other Etx vaccine polypeptides and polypeptides which are candidates for use as vaccines. For example, the following publications disclose polypeptides which have been shown to reduce toxicity towards MCDK cells and may be further improved by modifying domain III: Kang J et al. (2017) Hum Vaccin Immunother 13: 1598-1608 describes a proposed human vaccine with a mutation, F199, in domain 1; Yao et al. (2016) Sci Rep. 6: 24162 describes a Y196 mutation in domain 1; Li et al. (2013) Hum Vaccin Immunother 9: 2386-92 describes F199E and H106P mutants (F199 is in domain 1 and H106 is in domain 2); Oyston et al. (1998) Microbiology 144:333-41 describes a H106P mutant and also mentions H149P mutant as non-toxic (H106 is in domain 2); Dorca-Arévalo et al. (2014) PLoS One 9:e102417 describes V56C/F118C and H106P mutations as being non-toxic to MDCK cells (V56 is in domain 2 and F118 is in domain 2).

It was further surprisingly found that the Y30A-Y196A double mutant showed different toxicity results depending on the species from which the MAL is derived. For example, the double mutant was only marginally less toxic towards CHO cells expressing sheep MAL, but was more toxic to CHO cells expressing human MAL compared to wild type Etx. However, in CHO cells expressing dog MAL, the mutant was markedly less toxic. This finding suggests that MAL from different species interacts differently with Etx, indicating that MDCK cells may not be a good model for testing the toxicity of Etx vaccine candidates and that CHO cells expressing MAL might be a better model for such testing. Therefore, according to a further aspect of the present invention, there is provided use of (CHO) cells expressing MAL as a model in the testing for toxicity of epsilon vaccine candidates.

Table 3 below shows examples of mutations introduced into domain III of a Y30A-Y196A double mutant.

TABLE 3
Y30A-Y196A FURTHER MUTATIONS

| | Mutation Positions (counted | |
|---|---|---|
| | from residue 1 of SEQ ID NO: | |
| SEQ ID NO of | 65) | |
| trypsin | X may be any other amino | |
| activated | acid than the one shown | |
| recombinant | preceding the position | |
| epsilon toxin | number | Mutation Example |
| SEQ ID NO: 16 | Y30X + Y196X | Y30A + Y196A |
| SEQ ID NO: 17 | Y30X + Y196X + H149X | Y30A + Y196A + H149A |
| SEQ ID NO: 18 | Y30X + Y196X + V72X | Y30A + Y196A + V72F |
| SEQ ID NO: 19 | Y30X + Y196X + F92X | Y30A + Y196A + F92A |
| SEQ ID NO: 20 | Y30X + Y196X + V166X | Y30A + Y196A + V166A |
| SEQ ID NO: 21 | Y30X + Y196X + A168X | Y30A + Y196A + A168F |
| SEQ ID NO: 22 | Y30X + Y196X + | Y30A + Y196A + |
| | H149X + V72X | H149A + V72F |
| SEQ ID NO: 23 | Y30X + Y196X + | Y30A + Y196A + |
| | H149X + F92X | H149A + F92A |
| SEQ ID NO: 24 | Y30X + Y196X + | Y30A + Y196A + |
| | H149X + V166X | H194A + V166A |
| SEQ ID NO: 25 | Y30X + Y196X + | Y30A + Y196A + |
| | H149X + A168X | H149A + A168F |
| SEQ ID NO: 26 | Y30X + Y196X + | Y30A + Y196A + |
| | V72X + F92X | V72F + F92A |
| SEQ ID NO: 27 | Y30X + Y196X + | Y30A + Y196A + |
| | V72X + V166X | V72F + V166A |
| SEQ ID NO: 28 | Y30X + Y196X + | Y30A + Y196A + |
| | V72X + A168X | V72F + A168F |
| SEQ ID NO: 29 | Y30X + Y196X + | Y30A + Y196A + |
| | V92X + V166X | V92F + V166A |
| SEQ ID NO: 30 | Y30X + Y196X + | Y30A + Y196A + |
| | V92X + A168X | V92F + A168F |
| SEQ ID NO: 31 | Y30X + Y196X + | Y30A + Y196A + |
| | V166X + A168X | V166A + A168F |
| SEQ ID NO: 32 | Y30X + Y196X + H149X + | Y30A + Y196A + H149A + |
| | V72X + F92X | V72F + F92A |
| SEQ ID NO: 33 | Y30X + Y196X + H149X + | Y30A + Y196A + H149A + |
| | V72X + V166X | V72F + V166X |
| SEQ ID NO: 34 | Y30X + Y196X + H149X + | Y30A + Y196A + H149A + |
| | V72X + A168X | V72F + A168X |
| SEQ ID NO: 35 | Y30X + Y196X + H149X + | Y30A + Y196A + H149A + |
| | F92X + V166X | F92A + V166A |
| SEQ ID NO: 36 | Y30X + Y196X + H149X + | Y30A + Y196A + H149A + |
| | F92X + A168X | F92A + A168F |
| SEQ ID NO: 37 | Y30X + Y196X + H149X + | Y30A + Y196A + H149A + |
| | V166X + A168X | V166A + A168F |
| SEQ ID NO: 38 | Y30X + Y196X + V72X + | Y30A + Y196A + V72F + |
| | F92X + V166X | F92A + V166A |
| SEQ ID NO: 39 | Y30X + Y196X + V72X + | Y30A + Y196A + V72F + |
| | F92X + A168X | F92A + A168F |
| SEQ ID NO: 40 | Y30X + Y196X + V72X + | Y30A + Y196A + V72F + |
| | V166X + A168X | V166A + A168F |
| SEQ ID NO: 41 | Y30X + Y196X + F92X + | Y30A + Y196A + F92A + |
| | V166X + A168X | V166A + A168F |
| SEQ ID NO: 42 | Y30X + Y196X + H149X + | Y30A + Y196A + H149A + |
| | V72X + F92X + V166X | V72F + F92A + V166A |
| SEQ ID NO: 43 | Y30X + Y196X + H149X + | Y30A + Y196A + H149A + |
| | V72X + F92X + A168X | V72F + F92A + A168F |
| SEQ ID NO: 44 | Y30X + Y196X + V72X + | Y30A + Y196A + V72F + |
| | F92X + V166X + A168X | F92A + V166A + A168F |
| SEQ ID NO: 45 | Y30X + Y196X + H149X + | Y30A + Y196A + H149A + |
| | V72X + F92X + | V72F + F92A + |
| | V166X + A168X | V166A + A168F |
| SEQ ID NO: 46 | V72X | V72F |
| SEQ ID NO: 47 | F92X | F92A |
| SEQ ID NO: 48 | H149X | H149A |
| SEQ ID NO: 49 | V166X | V166A |
| SEQ ID NO: 50 | A168X | A168F |

The new sequences described herein (SEQ ID NOs 18 to 50 of Table 3) are therefore genetic toxoids suitable for incorporation into next generation enterotoxaemia or demyelinating disease vaccines; and vaccines against developing a disease caused by or associated with Clostridium perfringens and/or Etx. In particular, SEQ ID NO: 21 is suitable.

The mutations shown in Table 3 are described as comprised in a trypsin activated recombinant Etx (SEQ ID NO:13 or SEQ ID NO: 65), but the same mutations may also be comprised at equivalent positions in a full length Etx polypeptide (SEQ ID NO: 11) or at equivalent positions in the full length recombinant Etx polypeptide (SEQ ID NO: 15); or the mutations shown in Table 3 may be comprised at equivalent positions in a sequence having at least 60%, 65%, 70%, 75%, 80%, 85%, 90%, 95%, 96%, 97%, 98% or 99% sequence identity to SEQ ID NOs 11, 12, 13, 14, 15 or 65.

Any of the mutations described herein may be comprised in a sequence having at least 60%, 65%, 70%, 75%, 80%, 85%, 90%, 95%, 96%, 97%, 98% or 99% sequence identity to SEQ ID NOs 11, 12, 13, 14, 15 or 65.

The polypeptides of SEQ ID NOs 18 to 50 may have equal or similar or reduced toxicity to SEQ ID NO:14, or SEQ ID NO:14 lacking the first 2 N-terminal amino acid residues, and/or be non-toxic.

The polypeptide according to the invention may include a sequence of at least 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310 or 315 contiguous amino acids from any of SEQ ID NO: 11, SEQ ID NO: 12, SEQ ID NO: 13, SEQ ID NO: 14, SEQ ID NO: 15 or SEQ ID NO: 65 and comprising a double mutation at positions equivalent to positions 30-196 of SEQ ID NO:65 and additionally at least one, two or more mutations at positions equivalent to positions 72, 92, 149, 166 and 168. The numbering of the mutation positions is as described herein. The mutations may comprise a substitution or deletion.

The polypeptide according to the invention may include a sequence of at least 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 261, 262 or 263 contiguous amino acids from any one of SEQ ID NOs 18 to 50 from Table 3 and comprising or consisting of the mutations indicated for the relevant SEQ ID NO in Table 3.

The inclusion of one of amino acid sequences SEQ ID NOs 4 to 10 in the polypeptide according to the invention has the result that the sequence included in the polypeptide is identical to the equivalent (i.e., corresponding) sequence from any of SEQ ID NO: 11, SEQ ID NO: 12, SEQ ID NO: 13, SEQ ID NO: 14, SEQ ID NO: 15 or SEQ ID NO: 65, apart from the mutated position included as “X” within one or SEQ ID NOs 4 to 10.

The polypeptide according to the invention may have at least 60%, 65%, 70%, 75%, 80%, 85%, 90%, 95%, 96%, 97%, 98%, 99% or 100% sequence identity to any one of SEQ ID NOs 18 to 50 from Table 3 and comprising or consisting of the mutations indicated for the relevant SEQ ID NO.

The level of toxicity may be determined as described herein, in vitro or in vivo. For example, by use of a cell-based assay, such as MDCK.2 cell-based LDH assay, ACHN cells, the use of Chinese hamster ovary (CHO) cells (for example, CHO cells expressing green fluorescent protein (GFP), Human MAL (CHO-hMAL), sheep MAL (CHO-sMAL), dog MAL (CHO-dMAL) etc.). Toxicity studies may also be carried out in vivo, for example, in mice or any other suitable animal.

The polypeptides of the invention provide protection, when administered to a subject such as a human or a non-human animal such as lamb, sheep, goat, pig, cows, horses or rabbits, to the subject from developing a disease (such as enterotoxemia) caused by infection by Clostridium perfringens and/or caused by the presence of active epsilon toxin (or associated with the presence of Clostridium perfringens or the toxin) and/or a condition associated with demyelination. Such protection may be partial, whereby the probability of an individual subject within a population of developing disease is reduced, or complete, whereby the subject will not develop disease (i.e., the probability of developing disease caused by or associated with C. perfringens and/or by Etx is 0%).

The term “subject” as used throughout this specification, in relation to any aspect of the invention, indicates any human or animal individual, including (but not limited to) a cat, dog or horse, or a ruminant animal, cow, sheep, goat or pig. The human or animal may be a human or animal exhibiting symptoms of a demyelinating disease, for example enterotoxemia (ET), multiple sclerosis (MS), clinically definite MS (CDMS), clinically isolated syndrome (CIS), neuromyelitis optica spectrum disorder (NMOSD), optic neuritis (ON) or neuromyelitis optica (NMO), myelitis, transverse myelitis (TM), a disease or condition characterised by the increase or presence of antibodies against aquaporin-4 (AQP-4) and/or astrocyte damage, and acute disseminated encephalomyelitis (ADEM).

As outlined further below, sequence identity may be determined using the Needleman-Wunsch Global Sequence Alignment Tool available from the National Center for Biotechnology Information (NCBI), Bethesda, Maryland, USA, for example via http://blast.ncbi.nlm.nih.gov/Blast.cgi, using default parameter settings. When comparing the level of sequence identity to (for example) SEQ ID NO: 65, this typically should be done relative to the whole length of SEQ ID NO: 65, to avoid short regions of high identity overlap resulting in a high overall assessment of identity (i.e., a global alignment method is used). For example, a short polypeptide fragment having, for example, five amino acids might have a 100% identical sequence to a five amino acid region within the whole of SEQ ID NO:65, but this does not provide a 100% amino acid identity unless the fragment forms part of a longer sequence which also has identical amino acids at the other positions equivalent to positions in SEQ ID NO:65. For example, SEQ ID NO:11 is 79% identical at a global level to SEQ ID NO:65; positions 46-305 of SEQ ID NO:11 are 100% identical to positions 1-260 of SEQ ID NO:65, with positions 1-45 and 306-328 of SEQ ID NO:11 being absent from SEQ ID NO:65. SEQ ID NO:65 is the sequence equivalent to (or corresponding to) positions 46-305 of SEQ ID NO:11 and positions 46-305 of SEQ ID NO:11 are equivalent to (or correspond to) SEQ ID NO:65.

Therefore, the skilled person is readily able to determine equivalent positions between two sequences, by aligning sequences to achieve maximum identical amino acids at as many positions as possible, for example by using a global sequence alignment program such as is available via http://blast.ncbi.nlm.nih.gov/Blast.cgi, discussed further below.

The present invention also encompasses polypeptides comprising variants of the polypeptides and methods utilising these variant polypeptides. As used herein, a “variant” means a polypeptide in which the amino acid sequence differs from the base sequence from which it is derived in that one or more amino acids within the sequence are substituted for other amino acids. The variant is a functional variant, in that the functional characteristics of the polypeptide from which the variant is derived are maintained. For example, the variant polypeptide may have a similar ability to bind an antibody capable of binding to a non-variant polypeptide (such as, by way of non-limiting example, any of SEQ ID NOs 18 to 50 of Table 3). In particular, any amino acid substitutions, additions or deletions must not alter or significantly alter the tertiary structure of one or more epitopes contained within the polypeptide from which the variant is derived, so that the variant polypeptide retains the ability to bind to an antibody which binds to SEQ ID NO:65 or 14. The skilled person is readily able to determine appropriate functional variants and to determine the tertiary structure of an epitope and any alterations thereof, without the application of inventive skill.

Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

| CLASS | AMINO ACID EXAMPLES |
|---|---|
| Nonpolar: | Ala, Val, Leu, Ile, Pro, Met, Phe, Trp |
| Uncharged | Gly, Ser, Thr, Cys, Tyr, Asn, Gln |
| polar: | |
| Acidic: | Asp, Glu |
| Basic: | Lys, Arg, His. |

As is well known to those skilled in the art, altering the primary structure of a polypeptide by a conservative substitution may not significantly alter the activity of that polypeptide because the side-chain of the amino acid which is inserted into the sequence may be able to form similar bonds and contacts as the side chain of the amino acid which has been substituted out. This is so even when the substitution is in a region which is critical in determining the polypeptide's conformation.

As mentioned above, non-conservative substitutions are possible provided that these do not disrupt the tertiary structure of an epitope within the polypeptide, for example, which do not interrupt the immunogenicity (for example, the antigenicity) of the polypeptide.

Broadly speaking, fewer non-conservative substitutions will be possible without altering the biological activity of the polypeptide. As mentioned above, variants may suitably be at least about 60%, 65%, 70%, 75%, 80%, 85%, 90%, 95%, 96%, 97%, 98%, 99% identical to the base sequence.

As already briefly mentioned, sequence identity between amino acid sequences can be determined by comparing an alignment of the sequences. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences, to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties. As mentioned above, the percentage sequence identity should be determined using the Needleman-Wunsch Global Sequence Alignment tool, publicly available via http://blast.ncbi.nlm.nih.gov/Blast.cgi, using default parameter settings. The Needleman-Wunsch algorithm was published in J. Mol. Biol. (1970) vol. 48:443-53.

A further aspect of the invention provides a polynucleotide having a nucleic acid sequence which encodes for a polypeptide according to the first aspect of the invention. The invention also encompasses variant nucleic acids encoding the polypeptides of the invention. The term “variant” in relation to a nucleic acid sequence means any substitution of, variation of, modification of, deletion of, or addition of one or more nucleic acid(s) from or to a polynucleotide sequence, providing the resultant polypeptide sequence encoded by the polynucleotide exhibits at least the same properties as the polypeptide encoded by the basic sequence. The term therefore includes allelic variants and also includes a polynucleotide (a “probe sequence”) which substantially hybridises to the polynucleotide sequence of the present invention. Such hybridisation may occur at or between low and high stringency conditions. In general terms, low stringency conditions can be defined as hybridisation in which the washing step takes place in a 0.330-0.825 M NaCl buffer solution at a temperature of about 40-48 °C below the calculated or actual melting temperature (Tm) of the probe sequence (for example, about ambient laboratory temperature to about 55 °C), while high stringency conditions involve a wash in a 0.0165-0.0330 M NaCl buffer solution at a temperature of about 5-10 °C below the calculated or actual Tm of the probe sequence (for example, about 65 °C). The buffer solution may, for example, be SSC buffer (0.15M NaCl and 0.015M tri-sodium citrate), with the low stringency wash taking place in 3×SSC buffer and the high stringency wash taking place in 0.1×SSC buffer. Steps involved in hybridisation of nucleic acid sequences have been described for example in Sambrook et al. (2001; “Molecular Cloning: a laboratory manual”, 3^{rd} Edition, Cold Spring Harbor Laboratory Press, New York).

Polypeptides and nucleic acids of the invention may be prepared synthetically using conventional synthesisers. Alternatively, they may be produced using recombinant DNA technology and may be incorporated into a suitable expression vector, which is then used to transform a suitable host cell, such as a prokaryotic cell such as E. coli. The transformed host cells are cultured and the polypeptide isolated therefrom.

Therefore, the invention also provides a vector comprising such a polynucleotide. This includes recombinant constructs comprising one or more of the nucleic acid molecules described above. The constructs comprise a vector, such as a plasmid or viral vector, into which a nucleic acid molecule of the invention has been inserted, in a forward or reverse orientation. In a preferred aspect of this embodiment, the construct further comprises regulatory sequences, including, for example, a promoter operably linked to the sequence. Large numbers of suitable vectors and promoters are known to those of skill in the art and are commercially available. Appropriate cloning and expression vectors for use with prokaryotic and eukaryotic hosts are also described in Sambrook, et al.

A further aspect of the invention provides a cell comprising any of the polypeptide, polynucleotide or vector according to the invention. For example, a suitable cell may be a Salmonella cell, such as a Salmonella enterica cell, in some embodiments from the serovar typhimurium. The Salmonella may be an attenuated strain. Strains λ⁸⁹¹⁴ and λ⁹²⁴1 may optionally be employed. For example, a suitable system is described in Kulkarni et al. (2008, Vaccine vol. 26, 4194-4203). Preferably the host cell is not a stem cell, especially not a human stem cell such as a human embryonic stem cell.

A further aspect of the invention provides an affinity reagent which is capable of binding to one of the polypeptides according to the first aspect of the invention and facilitating an immune response in the body of an individual to which the affinity reagent is administered. For example, the affinity reagent may be an antibody which may be a monoclonal antibody or a synthetic antibody, an Affibody® molecule or other antibody mimetic, an aptamer, a protein scaffold or a major histocompatibility complex (MHC) protein or portion thereof. The affinity reagent may be an antibody raised against a polypeptide according to the first aspect of the invention.

A further aspect of the invention provides a subunit or conjugate vaccine comprising a polypeptide according to the first aspect of the invention. For example, this may be in the form of a fusion protein and/or in the form of a recombinant viral vaccine.

A further aspect of the invention provides a method for the preparation of a vaccine, comprising adapting an Etx polypeptide or a vaccine or vaccine candidate comprising an Etx polypeptide by modifying domain III relative to wild type Etx polypeptide SEQ ID NO:65. A further aspect provides a vaccine composition prepared by a method comprising the aforementioned method.

The vaccine or vaccine candidate for improvement (by modification of domain III compared to a wild type polypeptide) may be selected from any of the following, which after modification of domain III show reduced toxicity compared to the original vaccine or candidate vaccine. For example, the following publications disclose polypeptides which have been shown to reduce toxicity towards MCDK cells and may be further improved by modifying domain III: Kang J et al. (2017) Hum Vaccin Immunother 13: 1598-1608 describes a proposed human vaccine with mutation F199 in domain 1; Yao et al. (2016) Sci Rep. 6: 24162 describes a Y196 mutation in domain 1; Li et al. (2013) Hum Vaccin Immunother 9: 2386-92 describes F199E and H106P mutants (F199 is in domain 1 and H106 is in domain 2); Oyston et al. (1998) Microbiology 144:333-41 describes a H106P mutant and also mentions H149P mutant as non-toxic (H106 is in domain 2); Dorca-Arévalo et al. (2014) PLoS One 9:e102417 describes V56C/F118C and H106P mutations as being non-toxic to MDCK cells (V56 is in domain 2 and F118 is in domain 2).

A further aspect provides an immunotherapy composition comprising a polypeptide, polynucleotide, vector, affinity reagent, subunit vaccine and/or conjugate vaccine according to preceding aspects of the invention, in a pharmaceutically acceptable formulation. For example, the immunotherapy composition may be a vaccine composition comprising a polypeptide according to the first aspect of the invention and an adjuvant. The composition may further comprise excipients and/or diluents appropriate for the means by which the composition is to be administered to a subject in need of treatment or vaccination against developing disease caused by C. perfringens and/or Etx. Selection of appropriate components is within the routine capability of the skilled person without the application of inventive activity.

For example, the immunotherapy composition of the invention may conveniently be formulated using a pharmaceutically acceptable excipient or diluent, such as, for example, an aqueous solvent, non-aqueous solvent, non-toxic excipient, such as a salt, preservative, buffer and the like. Examples of non-aqueous solvents are propylene glycol, polyethylene glycol, vegetable oil and injectable organic esters such as ethyloleate. Aqueous solvents include water, alcoholic/aqueous solutions, saline solutions, parenteral vehicles such as sodium chloride, Ringer's dextrose, etc. Preservatives include antimicrobial, anti-oxidants, chelating agents and inert gases. The pH and exact concentration of the various components the vaccine composition are adjusted according to routine skills.

Optionally, the immunotherapy formulation may include a carrier. Commonly used carrier molecules are bovine serum albumin (BSA), keyhole limpet hemocyanin (KLH), ovalbumin, mouse serum albumin, rabbit serum albumin and the like. Synthetic carriers may be used and are readily available. Means for conjugating peptides to carrier proteins are well known in the art and include glutaraldehyde, m-maleimidobenzoyl-N-hydroxysuccinimide ester, carbodiimide and bis-biazotized benzidine.

In certain situations, it may also be desirable to formulate the immunotherapy composition to comprise an adjuvant to enhance the immune response. Such adjuvants include all acceptable immunostimulatory compounds such as, for example, a cytokine, toxin, or synthetic composition. Commonly used adjuvants include aluminium hydroxide, aluminium phosphate, calcium phosphate, Freund's adjuvants and Quil-A saponin. An adjuvant provided by SEPPIC Inc (New Jersey, USA) as a Montanide™ adjuvant, for example Montanide™ ISA 61VG, may also be a suitable adjuvant. The inventors found that the use of Montanide™ ISA 61VG adjuvant resulted in the induction of better antibody responses compared to the use of an alhydrogel adjuvant. In addition to adjuvants, it may be desirable to co-administer biologic response modifiers (BRM) with the peptide or variant or derivative to down regulate suppressor T cell activity.

Possible vehicles for administration of the immunotherapy formulation include liposomes. Liposomes are microscopic vesicles that consist of one or more lipid bilayers surrounding aqueous compartments. Liposomes are similar in composition to cellular membranes and, as a result, liposomes generally can be administered safely and are biodegradable. Techniques for preparation of liposomes and the formulation (e.g., encapsulation) of various molecules, including peptides and oligonucleotides, with liposomes are well known.

Depending on the method of preparation, liposomes may be unilamellar or multilamellar and can vary in size with diameters ranging from 0.02 μm to greater than 10 μm. Liposomes can also adsorb to virtually any type of cell and then release the encapsulated agent. Alternatively, the liposome fuses with the target cell, whereby the contents of the liposome empty into the target cell. Alternatively, an absorbed liposome may be endocytosed by cells that are phagocytic. Endocytosis is followed by intralysosomal degradation of liposomal lipids and release of the encapsulated agents. In the present context, the polypeptide according to the invention can be localized on the surface of the liposome, to facilitate antigen presentation without disruption of the liposome or endocytosis. Irrespective of the mechanism or delivery, however, the result is the intracellular disposition of the associated polypeptide.

Liposomal vectors may be anionic or cationic. Anionic liposomal vectors include pH sensitive liposomes which disrupt or fuse with the endosomal membrane following endocytosis and endosome acidification. Cationic liposomes are preferred for mediating mammalian cell transfection in vitro, or general delivery of nucleic acids, but are used for delivery of other therapeutics, such as peptides.

Other suitable liposomes that are used in the methods of the invention include multilamellar vesicles (MLV), oligolamellar vesicles (OLV), unilamellar vesicles (UV), small unilamellar vesicles (SUV), medium-sized unilamellar vesicles (MIN), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV). Techniques for preparing these liposomes are well known in the art.

Other forms of delivery particle, for example, microspheres and the like, also are contemplated for delivery of the peptide epitopes or polyepitopes.

Alternatively, nucleic acid-based vaccines may be produced that comprise nucleic acid, such as, for example, DNA or RNA, encoding the immunologically active peptide epitope or polyepitope and cloned into a suitable vector (e.g., vaccinia, canarypox, adenovirus, or other eukaryotic virus vector).

Alternatively, the polypeptide may be administered in the form of a cellular vaccine via the administration of autologous or allogeneic APCs or dendritic cells that have been treated in vitro so as to present the peptide on their surface. Salmonella enterica or Escherichia coli strains harbouring mutations which reduce their virulence and allow them to colonise a host animal without causing disease might be used to deliver vaccine antigens, especially for administration to non-human animals. The bacteria used might include strains which are already used as vaccine in livestock where the attenuating lesion is not fully characterised. In addition, strains in which mutations have been deliberately introduced into the bacterium to rationally attenuate virulence could be used to deliver the polypeptide, as described in WO2013/144636. The antigen might also be delivered as a naked DNA vaccine where the gene encoding the epsilon toxoid is cloned into a mammalian expression vector and expressed from a eukaryotic promoter.

One of the most widely studied classes of attenuated Salmonella used as carriers of foreign antigens are auxotrophs. For example, genetically defined mutants of the aroA gene, encoding 5-eno/pyruvylshikimate-3-phosphate synthase, have been constructed in both S. enterica var. Typhimurium and var. Typhi. These mutants are attenuated and immunogenic in mice. Examples of other auxotrophic mutants include Salmonella with deletions in the genes involved in the purine biosynthetic pathway. Another well-studied group of attenuated Salmonella are mutants that have defined deletions in genes involved in the regulation of Salmonella virulence. For example, mutations in genes encoding adenylate cyclase (cya) and camp receptor protein (crp) affect the expression of genes involved.

In one embodiment, the immunotherapy composition may be included in a foodstuff (i.e., a food material suitable for consumption by a human or an animal) comprising a polypeptide and/or a polynucleotide and/or a vector and/or a cell and/or a subunit vaccine and/or vaccine composition according to preceding aspects of the invention. This may, in non-limiting examples, be in the form of pellets, crumbs or a mash which may further comprise, again for example only, grain, grass and/or protein components. The composition may also be included in drinking liquids and/or administered via a spray into the atmosphere surrounding the animal which is, consequently, inhaled by the animal.

If the vaccine composition is for administration to a human subject, it may be in a form suitable for administration orally (e.g. in a dietary supplement) and/or parenterally, for example, by injection, inhalation, or by transdermal administration via a patch, lotion or gel. The particular forms outlined above are also generally useful for administration to a human subject.

A polypeptide, polynucleotide, vector, subunit vaccine, conjugate vaccine, antibody, affinity reagent, vaccine composition and/or immunotherapy composition according to the invention may be for use in a method of treating or vaccinating a subject against developing a disease caused by Clostridium perfringens and/or Etx, the disease for example involving accumulation in the subject's bloodstream of epsilon toxin which may be released by C. perfringens, particularly active epsilon toxin lacking the N- and C-termini of the full length prototoxin and/or for use in treating or vaccinating a subject against a condition associated with demyelination. A polypeptide, polynucleotide, vector, subunit vaccine, conjugate vaccine, affinity reagent, vaccine composition and/or immunotherapy composition according to the invention may be for use in a method of treatment or vaccinating a subject against developing a disease associated with infection by Clostridium perfringens or associated with the presence of Etx or condition associated with demyelination. According to one embodiment, the disease is enterotoxemia or a demyelinating disease, such as multiple sclerosis, neuromyelitis optica (NMO), optic neuritis (ON) or myelitis.

The invention also provides a method of vaccinating a subject against developing a disease caused by Clostridium perfringens and/or caused by the epsilon toxin, especially the active toxin; or associated with infection by Clostridium perfringens or associated with the presence of Etx or a condition associated with demyelination, the method comprising administering to a subject a polypeptide, polypeptide, polynucleotide, vector, subunit vaccine, conjugate vaccine, affinity reagent, vaccine composition and/or immunotherapy reagent according to the invention (for example, in a protective amount). The subject may be a human or a non-human animal. The non-human animal may be a horse or ruminant animal, such as a sheep, pig or goat, or a bovine animal such as a domestic cow or a companion animal, such as a dog, cat or rabbit. Young animals, such as lambs, piglets, kids and calves are also included.

A “protective amount” is an amount sufficient to induce an immune response in the subject, such that the probability of the subject developing a disease caused by C. perfringens, for example caused by (or associated with the presence of) epsilon toxin, especially active toxin, is reduced or removed. For example, antibodies capable of binding to SEQ ID NO:65 and/or 14 may be detectable after the administration, where such antibodies were not detectable prior to the administration, or only detectable at lower concentrations than after administration.

The invention also provides a kit comprising a polypeptide, polynucleotide, vector, subunit vaccine, conjugate vaccine, affinity reagent, vaccine composition and/or immunotherapy reagent according to the invention, the kit having uses, for example, in methods of treatment or vaccinating a subject against developing a disease caused by infection with Clostridium perfringens and/or Etx; or in methods of treatment or vaccinating a subject against developing a disease associated with infection by Clostridium perfringens or associated with the presence of Etx or a condition associated with demyelination. The kit may comprise means for administering the polypeptide, polynucleotide, vector, subunit vaccine, conjugate vaccine, affinity reagent, vaccine composition and/or immunotherapy reagent to an individual. For example, the kit may comprise one or more buffer reagents or diluents and/or one or more administration devices such as a syringe or other injection device. The kit may alternatively or additionally comprise instructions enabling a user to carry out a method of treatment or vaccinating a subject against developing a disease caused by or associated with Clostridium perfringens and/or Etx and/or a demyelinating disease, such as enterotoxemia (ET), multiple sclerosis (MS), clinically definite MS (CDMS), clinically isolated syndrome (CIS), neuromyelitis optica spectrum disorder (NMOSD), neuromyelitis optica (NMO), optic neuritis (ON), myelitis, transverse myelitis (TM), a disease or condition characterised by the increase or presence of antibodies against aquaporin-4 (AQP-4) and/or astrocyte damage, and acute disseminated encephalomyelitis (ADEM).

A disease caused by or associated with Clostridium perfringens and/or (active) epsilon toxin, as mentioned herein may be, for example, enterotoxemia including pre-disease symptoms such as systemic lesions and enterocolitis. Other symptoms may include oedema of the main target organs of the kidneys and brain and damage to vascular endothelial cells. The terminal phase of enterotoxaemia is characterized by severe neurological disorders that include opisthotonus, seizures and agonal struggling. The disease may be a demyelinating disease, such as multiple sclerosis (MS), clinically definite MS (CDMS), clinically isolated syndrome (CIS), neuromyelitis optica spectrum disorder (NMOSD), neuromyelitis optica (NMO), optic neuritis (ON), myelitis, transverse myelitis (TM), a disease or condition characterised by the increase or presence of antibodies against aquaporin-4 (AQP-4) and/or astrocyte damage, and acute disseminated encephalomyelitis (ADEM).

Throughout the description and claims of this specification, the words “comprise” and “contain” and variations of the words, for example “comprising” and “comprises”, mean “including but not limited to” and do not exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

### BRIEF DESCRIPTION OF THE FIGURES

Embodiments of the invention will now be described, by way of example only, with reference to FIGS. 1-5 in which:

FIG. 1 is a schematic representation of recombinant epsilon protoxin (P-Etx), with a N-terminal PelB leader peptide in place of the 13 amino acids N-terminal peptide sequence and with a C-terminal His-tag to aid purification (the amino acid sequences around the processing sites are also shown);

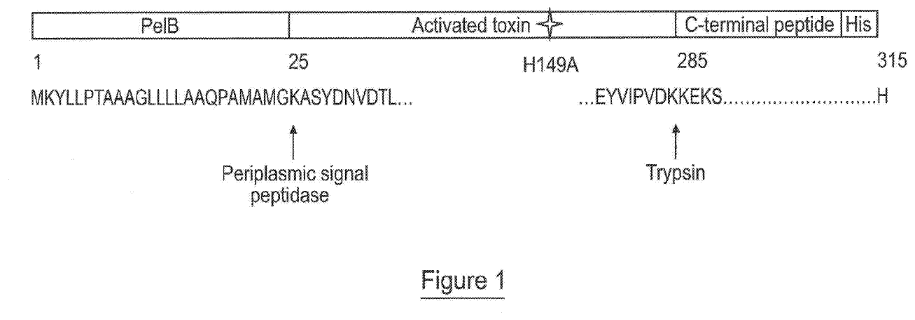

FIG. 2 shows the Etx amino acid sequence with positions of the signal sequence (residues 1-32); N-terminal pro-peptide (residues 33-45); C-terminal pro-peptide (residues 305-328); and mutated residues Y30A (Y43); V72F; F92A; H129A; V166A, A168F; Y196A(Y209), with the original unmutated amino acids shown under the mutation position;

FIG. 3A shows 5 residues flanking a glycan (β-octyl-glucoside) binding site in domain III of Etx (a suggested second receptor binding site) identified for mutagenesis: V72, F92, H149, V166, A168. Locations of residues previously mutated (shown in dark grey and circled (top); Y30 and Y196) and residues in the β-octyl-glucoside binding cleft (V72, F92, H149, V166, A168; shown in pale grey and circled (bottom);

FIG. 3B shows SDS-PAGE gel of purified proteins tested in this study. Using a plasmid which encoded the Y30A-Y196A variant form of Etx, additional mutations V72, F92, H149, V166 and A168 were introduced. These residues were mutated to alanine (H149, F92, V166) or phenylalanine (A168, V72) and the his-tagged proteins encoded by the mutated genes were expressed in E. coli and purified;

FIG. 4 shows the ability of WHO standard antitoxin (5 IU/ml) or rabbit sera raised against genetic toxoids, to displace binding of a neutralising monoclonal antibody in a competitive ELISA. The sera were diluted as indicated in the legend before testing. Data shown is the mean of two assays with SEM bars shown.

FIG. 5 shows the melting temperature (Tm) of wild type and variant proteins. Thermostability of Etx prototoxins was determined by the Boltzmann method using the Protein Thermal Shift software (Applied Biosystems). Results represent the mean and standard deviation of triplicate samples.

FIG. 6 show the results of the treatment of human red blood cells with epsilon toxin (wild type Etx; Y30AY196A; Y30AY196A+H149A; Y30AY196A+A168F; Y30AY196A+F92A; Y30AY196A+V166A, and a Quad: Y30AY196+A168F+H149A). Y30AY196A+A168F, Y30AY196A+H149A and the quad were not haemolytic even when trypsin activated.

### ITEMS

The present invention will now be described with reference to the following items in which:
1. A method for preventing or treating a demyelinating condition selected from: enterotoxemia (ET), multiple sclerosis (MS), clinically definite MS (CDMS), clinically isolated syndrome (CIS), neuromyelitis optica spectrum disorder (NMOSD), optic neuritis (ON), neuromyelitis optica (NMO), myelitis, transverse myelitis (TM), a disease or condition characterised by the increase or presence of antibodies against aquaporin-4 (AQP-4) and/or astrocyte damage, and acute disseminated encephalomyelitis (ADEM) in a human or animal subject in need, comprising: administering to said subject a composition comprising an effective amount of an agent that directly or indirectly interferes with epsilon toxin (Etx) produced by Clostridium perfringens type B or type D bacterial strain, an Etx-binding receptor, or an interaction of Etx with its binding receptor so as to inhibit or suppress Etx modulated receptor signalling activities.
2. Method of item 1, wherein said agent is an inhibitor of Etx, such as an antibody or a functional component thereof.
3. Method of item 1, wherein said agent is an inhibitor or antagonist of an Etx-binding receptor.
4. Method of item 3, wherein said Etx-binding receptor is myelin and lymphocyte protein (MAL) or the hepatitis A virus cell receptor 1 proteins (HAVCR1).
5. Method of item 1, wherein said agent is a vaccine against Clostridium perfringens type B or type D bacterial strain, or the epsilon toxin (Etx) produced therefrom.
6. Method according to any preceding item, wherein said agent and comprises an epsilon toxin (Etx) polypeptide having reduced toxicity to cells expressing Myelin And Lymphocyte (MAL) protein and comprising a modified domain III compared to wild type Etx polypeptide SEQ ID NO:65, wherein said reduced toxicity is relative to SEQ ID NO: 65 and/or SEQ ID NO: 14 and wherein said Etx polypeptide is capable of binding at least one antibody which binds to a sequence represented by SEQ ID NO:65 and/or SEQ ID NO:14.
7. Method of item 6, wherein said modified domain III is a modification in the glycan (β-octyl-glucoside) binding site of domain III.
8. Method according to item 6 or 7, wherein said modified domain III comprises one or more mutations of the amino acids within the amino acid sequences making up domain III as represented by SEQ ID NOs 1, 2 and 3.
9. Method according to any one of items 6 to 8, comprising one or more of the following: SEQ ID NO: 10, SEQ ID NO: 6, SEQ ID NO: 7, SEQ ID NO: 8 and SEQ ID NO: 9.
10. Method according to any one of items 6 to 9, comprising SEQ ID NO: 4 and/or SEQ ID NO: 5 and/or SEQ ID NO: 6.
11. Method according to any one of items 6 to 10, comprising at least the following sequences:
   i) SEQ ID NO: 4 and SEQ ID NO: 5;
and optionally in addition to (i)
   ii) SEQ ID NO: 6;
and
   iii) one or more of the following: SEQ ID NO:10, SEQ ID NO:7, SEQ ID NO:8 and SEQ ID NO:9.
12. Method according to any one of items 6 to 11, wherein said reduced toxicity is reduced compared to an Etx polypeptide comprising SEQ ID NO: 4 and SEQ ID NO: 5; or an Etx polypeptide comprising SEQ ID NO: 4, SEQ ID NO: 5 and SEQ ID NO: 6; or compared to a known Etx vaccine or Etx vaccine candidate.
13. Method according to any one of items 6 to 12 having at least 60% sequence identity to any one of SEQ ID NOs 18 to 50 and comprising or consisting of the mutation(s) indicated in Table 3 for the relevant SEQ ID NO.
14. A polynucleotide having a nucleic acid sequence which encodes for a polypeptide according to any preceding items.
15. A vector comprising a polynucleotide according to item 14 or an agent according to any of items 1-5.
16. A cell comprising an agent according to any of items 1-5, a polypeptide according to any of items 6-13 and/or a polynucleotide according to item 14 and/or a vector according to item 15.
17. A subunit or conjugate vaccine comprising an agent according to any one of items 1-5, a polypeptide according to any of item 6-13.
18. An affinity reagent which is capable of binding to an agent according to any of items 1-5, one of the polypeptides according to any of items 6-13 and facilitating an immune response in the body of an individual to which the affinity reagent is administered.
19. A method for the preparation of an immunotherapy composition, optionally a vaccine composition, comprising adapting an Etx polypeptide or a vaccine comprising an Etx polypeptide by modifying domain III relative to a wild type Etx polypeptide.
20. An immunotherapy or vaccine composition prepared by a method according to item 19.
21. An immunotherapy or vaccine composition comprising an agent according to any of items 1-5, a polypeptide according to any of item 6-13 and/or a polynucleotide according to item 14 and/or a vector according to item 15 and/or a cell according to item 16 and/or a subunit vaccine according to item 17 and/or affinity reagent according to item 14.
22. An immunotherapy composition, optionally a vaccine composition, according to item 20 or 21 which is a foodstuff for a human or animal.
23. An agent according to any of items 1-5, a polypeptide according to any of items 6-13 and/or a polynucleotide according to item 14 and/or a vector according to item 15 and/or a cell according to item 16 and/or a subunit or conjugate vaccine according to item 17 and/or an affinity reagent according to item 18 and/or an immunotherapy or vaccine composition according to any one of item 20-22 for use in a method of treating or vaccinating a subject against developing a disease caused by or associated with Clostridium perfringens and/or caused by or associated with (active) epsilon toxin and/or against a demyelinating disease.
24. A polypeptide according to item 23, wherein the disease is selected from enterotoxemia (ET), multiple sclerosis (MS), clinically definite MS (CDMS), clinically isolated syndrome (CIS), neuromyelitis optica spectrum disorder (NMOSD), optic neuritis (ON), neuromyelitis optica (NMO), myelitis, transverse myelitis (TM), a disease or condition characterised by the increase or presence of antibodies against aquaporin-4 (AQP-4) and/or astrocyte damage, and acute disseminated encephalomyelitis (ADEM).
25. A method of treating a subject having a disease caused by or associated with the presence of Clostridium perfringens and/or caused by or associated with the presence of (active) epsilon toxin and/or against a demyelinating disease, or a method for vaccinating a subject against developing such a disease, the method comprising administering to a subject an agent according to any of items 1-5, a polypeptide according to any of items 6-13 and/or a polynucleotide according to item 14 and/or a vector according to item 15 and/or a cell according to item 16 and/or a subunit vaccine according to item 17 and/or affinity reagent according to item 18 and/or a vaccine or immunotherapy composition according to any one of items 20-22, wherein the demyelinating condition is selected from: enterotoxemia (ET), multiple sclerosis (MS), clinically definite MS (CDMS), clinically isolated syndrome (CIS), neuromyelitis optica spectrum disorder (NMOSD), optic neuritis (ON), neuromyelitis optica (NMO), myelitis, transverse myelitis (TM), a disease or condition characterised by the increase or presence of antibodies against aquaporin-4 (AQP-4) and/or astrocyte damage, and acute disseminated encephalomyelitis (ADEM).
26. An agent, polypeptide, polynucleotide, vector, cell, affinity reagent, vaccine composition or immunotherapy composition according to any one of items 20-22, or a method according to item 25, wherein the subject is a ruminant animal, a horse, a companion animal or a human.
27. Use of MAL cells as a model in the testing for toxicity of epsilon vaccine candidates.
28. A kit comprising an agent according to any of items 1-5, polypeptide according to any of item 6-13 and/or a polynucleotide according to item 14 and/or a vector according to item 15 and/or a cell according to item 16 and/or a subunit vaccine according to item 17 and/or an affinity reagent according to item 18 and/or a vaccine or immunotherapy composition according to any one of items 20-22.
29. A polypeptide, polynucleotide, vector, cell, subunit vaccine, a conjugate vaccine, affinity reagent, vaccine composition or immunotherapy composition or method substantially as herein described.

### Examples

### Materials and Methods

### Chemicals

All chemicals were obtained from Sigma Chemical Co. (St. Louis, MO) unless otherwise specified.

### Expression and Purification of Recombinant Epsilon Toxin Wild Type and Mutants

The etxD gene, encoding epsilon prototoxin D from C. perfringens Type D strain NCTC 8346, was cloned into the expression vector pET-26b(+) (Merck, Darmstadt, Germany) with a N-terminal PelB leader peptide in place of the 13 amino acids N-terminal peptide sequence (residues KEISNTVSNEMSK) and with a C-terminal polyhistidine (6×His) tag to aid affinity purification of recombinant prototoxin (Bokori-Brown et al., (2013) Protein science: a publication of the Protein Society 22:650-9). Amino acid numbering corresponds to prototoxin without the 13 amino acid N-terminal peptide sequence. Mutations H149A, A168F, F92A, V166A and V72F (residues flanking the β-octyl-glucoside binding site) were introduced into the previously generated Y30A-Y196A mutant (Bokori-Brown et al. (2014) Vaccine 32:2682-7) using the QuickChange Lightning Site-Directed Mutagenesis Kit (Agilent Technologies, Inc. Santa Clara, US) according to the manufacturer's instructions (amino acid numbering corresponds to prototoxin without the 13 amino acids N-terminal peptide sequence). Primers used for site-directed mutagenesis are shown in Table 4 below. Recombinant proteins were expressed in E. coli Rosetta 2 (DE3) cells (Merck, Darmstadt, Germany) and grown in ZYM-5052 auto-induction medium (Studier FW (2005) Protein expression and purification 41:207-34) supplemented with 50 μg/mL kanamycin and 34 μg/mL chloramphenicol. Cells (100 mL) were grown at 37 °C for 3 h and cultured for a further 24 h at 20 °C, 300 rpm.

TABLE 4
PRIMERS USED FOR SITE DIRECTED MUTAGENESIS

| Amino | SEQ | |
|---|---|---|
| acid | ID | |
| change | NO | Primer sequence^{a} |
| Y30A | 51 | PRIMER ‘Y43A_FORWARD’: |
| | | GAAAGGAAGATATAATACAAAATATAATTACTTAAAGAGAATGGAAAAATATGCGCCTAATG |
| | | CTATGGCATATTTTGATAAGG |
| | 52 | PRIMER ‘Y43A_REVERSE’: |
| | | CCTTATCAAAATATGCCATAGCATTAGGCGCATATTTTTCCATTCTCTTTAAGTAATTATATT |
| | | TTGTATTATATCTTCCTTTC |
| Y196A | 53 | PRIMER ‘Y209A_FORWARD’: |
| | | GTGAATGGGGAGAGATACCTAGTGCGTTAGCTTTTCCTAGGGATGGTTA |
| | 54 | PRIMER ‘Y209A_REVERSE′: |
| | | TAACCATCCCTAGGAAAAGCTAACGCACTAGGTATCTCTCCCCATTCAC |
| H149A | 55 | PRIMER ‘H149A_FORWARD’: |
| | | CAAATACAAATACAAATACTAATTCAAAAGAAATTACTGCTAATGTCCCTTCACAAGATATA |
| | | CTA |
| | 56 | PRIMER ‘H149A_REVERSE’: |
| | | TAGTATATCTTGTGAAGGGACATTAGCAGTAATTTCTTTTGAATTAGTATTTGTATTTGTATT |
| | | TG |
| V72F | 57 | PRIMER ‘V72F_FORWARD’: |
| | | AGAACCATCAATGAATTATCTTGAAGATGTTTATTTTGGAAAAGCTCTCTTAAC |
| | 58 | PRIMER ‘V72F_REVERSE′: |
| | | GTTAAGAGAGCTTTTCCAAAATAAACATCTTCAAGATAATTCATTGATGGTTCT |
| F92A | 59 | PRIMER ‘F92A_FORWARD’: |
| | | TCTTAACTAATGATACTCAACAAGAACAAAAATTAAAATCACAATCAGCGACTTGTAAAAAT |
| | | ACTGATACAGTAAC |
| | 60 | PRIMER ′F92A_REVERSE’: |
| | | GTTACTGTATCAGTATTTTTACAAGTCGCTGATTGTGATTTTAATTTTTGTTCTTGTTGAGTA |
| | | TCATTAGTTAAGA |
| V166A | 61 | PRIMER ‘V166A_FORWARD’: |
| | | ATACTAGTACCAGCTAATACTACTGTAGAAGCGATAGCATATTTAAAAAAAGTTAATGTTAA |
| | | AG |
| | 62 | PRIMER ‘V166A_REVERSE’: |
| | | CTTTAACATTAACTTTTTTTAAATATGCTATCGCTTCTACAGTAGTATTAGCTGGTACTAGTA |
| | | T |
| A168F | 63 | PRIMER ‘A168F_FORWARD’: |
| | | GATATACTAGTACCAGCTAATACTACTGTAGAAGTAATATTTTATTTAAAAAAAGTTAATGTT |
| | | AAAGGAAATGTAAAGTTAG |
| | 64 | PRIMER ‘A168F_REVERSE’: |
| | | CTAACTTTACATTTCCTTTAACATTAACTTTTTTTAAATAAAATATTACTTCTACAGTAGTATT |
| | | AGCTGGTACTAGTATATC |

^{a}Underlined bases are the codons used for substitution. All primer sequences are shown in 5′ to 3′ orientation. Amino acid numbering corresponds to prototoxin without the N-terminal peptide sequence.

### Protein Purification

For protein purification, cells were harvested by centrifugation, lysed enzymatically using BugBuster™ Protein Extraction Reagent (Merck, Darmstadt, Germany), and Y30A-Y196A and its derivatives were purified by Ni-NTA chromatography columns (GE Healthcare Life Sciences, Little Chalfont, UK) according to the manufacturer's instructions. For buffer exchange and further sample clean up, prototoxin containing eluate was applied to a PD-10 Desalting Column (GE Healthcare Life Sciences, Little Chalfont, UK) and eluted in 10 mM phosphate buffer, 2.7 mM potassium chloride, 137 mM NaCl, pH 7.4. Protein concentrations were determined using the BCA assay (Fisher Scientific UK Ltd, Loughborough, UK).

For studies in mice, rabbits or sheep the proteins were treated to remove endotoxin using a commercially available kit containing a high capacity endotoxin removal resin (Pierce™ High Capacity Endotoxin Removal Spin Columns, Thermo Scientific) according to the manufacturer's instructions. Residual levels of endotoxin were measured using a quantitative chromogenic assay (Pierce LAL Chromogenic Endotoxin Quantitation kit, Thermo Scientific).

### Trypsin Activation

Purified recombinant epsilon prototoxin and its derivatives were activated with trypsin, TPCK treated from bovine pancreas (Sigma-Aldrich Company Ltd., Gillingham, UK), which removes the C-terminal peptide sequence. Trypsin was prepared in PBS and added to recombinant P-Etx at 1:100 (w/w) ratio and incubated at room temperature for 1 h. Protease Inhibitor Cocktail, EDTA-Free (Fisher Scientific UK Ltd, Loughborough, UK) was added to the digest to inhibit trypsin in the samples. Removal of the C-terminal peptide sequence was assessed by SDS-PAGE.

### SDS-PAGE Analyses

Protein purity was analysed by SDS-PAGE on 4-12% Bis-Tris NuPAGE gels (Invitrogen Ltd., Paisley, UK) using XCell SureLock™ Mini-Cell Electrophoresis System (Invitrogen Ltd., Paisley, UK) and NuPAGE MES SDS running buffer (Invitrogen Ltd., Paisley, UK). All samples were heated prior to loading at 70 °C for 10 min in NuPAGE LDS sample buffer (Invitrogen Ltd., Paisley, UK). Gels were typically run at 200 V for 45 min. After electrophoretic separation, proteins were visualized by SimplyBlue staining (Invitrogen Ltd., Paisley, UK). The Perfect Protein molecular weight standard (Merck, Darmstadt, Germany) was used as marker.

### Thermostability Assay

Thermostability was assessed by mixing purified protein (0.25 mg/mL) with 240× SYPRO Orange protein gel stain (Sigma-Aldrich Company Ltd., UK). Fluorescence was monitored using a StepOnePlus quantitative PCR machine (Applied Biosystems, USA) with a 1% thermal gradient from 25 °C to 99 °C. The fluorescence data obtained was analysed using the Protein Thermal Shift Software (Applied Biosystems, USA) to calculate the melting temperature (Tm) using the Boltzmann method. All measurements were performed in triplicate.

### Cell Culture

MDCK.2 cells (ATCC-LGC Standards, Teddington, UK) and ACHN cells (ECACC, Salisbury, UK) were routinely cultured in Eagle's Minimum Essential Medium (EMEM; ATCC-LGC Standards, Teddington, UK) supplemented with 10% Foetal Bovine Serum Gold (PAA, Pasching, Austria) at 37 °C in a humidified atmosphere of 95% air/5% CO₂. The culture medium was replaced every 2-3 days. Cells were routinely detached by incubation in trypsin/EDTA and split as appropriate (typically 1:6 dilutions).

Chinese hamster ovary (CHO) cells or CHO cells expressing green fluorescent protein (GFP)-tagged Human MAL (CHO-hMAL), sheep MAL (CHO-sMAL) or dog MAL (CHO-dMAL) were routinely cultured in Dulbecco's Modified Eagle's Medium/Ham's F12 (DMEM/F12) medium (Life Technologies) supplemented with 10% Foetal Bovine Serum at 37 °C in a humidified atmosphere of 95% air/5% CO₂. The culture medium was replaced every 2-3 days. Cells were routinely detached by incubation in trypsin/EDTA and split as appropriate (typically 1:6 dilutions).

### Cytotoxicity Assay

The cytotoxic activity of trypsin-activated toxins toward MDCK.2 cells was determined by measuring the amount of lactate dehydrogenase (LDH) released from the cytosol of lysed cells into the cell culture medium using the CytoTox 96 nonradioactive cytotoxicity assay kit (Promega UK, Southampton, UK) according to the manufacturer's protocol. In brief, a two-fold dilution series of each activated toxin (ranging from 1 μM to 0.15 nM) was prepared in PBS and added to cells seeded into 96-well plate (3×10⁴ cells/well). Following incubation at 37 °C for 3 h, cell culture medium (50 μL) was harvested from cell monolayers, transferred to a fresh 96-well enzymatic assay plate and 50 μL of reconstituted substrate mix was added to each well. The plate was incubated for 30 min at room temperature, protected from light. Absorbance was read at 490 nm using a Model 680 Microplate Reader (Bio-Rad). The absorbance values for each sample were normalized by subtracting the absorbance value obtained for the culture medium from untreated cells. The toxin dose required to kill 50% of the cell monolayer (CT₅₀) was determined by nonlinear regression analysis (GraphPad). All experiments were performed in triplicates with three technical replicates each.

### Immunisation of Rabbits

Groups of three New Zealand White rabbits were each immunized subcutaneously with 100 ug of Y30A-Y196A, Y30A-Y196A-A168F or Y30A-Y196A-H149A-A168F by Cambridge Research Biochemicals. Freund's Complete adjuvant was used for the initial immunisation and Freund's Incomplete adjuvant was use for 4 subsequent immunisations given at 14 day intervals. Blood was collected 7 days after booster dose 3 (day 49) and 7 days after booster dose 4 (day 63).

WHO International Standard C. perfringens epsilon horse antitoxin serum (CPEPAT) was obtained from the National Institute for Biological Standards and Control (NIBSC, South Mimms, UK).

Immunisation of Sheep with Y30AY196A+A168F Toxoid

Lambs were reared without vaccination against C. perfringens epsilon toxin and tested at intervals for the presence of antibody against epsilon toxin using Western blotting. After 12 months one group of 5 lambs received nothing, one group of 6 lambs received 200 μg of Y30AY196A+A168F toxoid adjuvanted (1:1) with Montanide™ ISA 61VG (Seppic, Paris, France), and a group of 5 lambs received 200 μg of the Y30AY196A+A168F toxoid adjuvanted with aluminium hydroxide gel (alhydrogel; Sigma-Aldrich, Poole UK; 0.25% w/v final concentration) adjuvant. We also immunised a group of 4 lambs, which had significant levels of pre-existing reactivity with epsilon toxin, with 200 μg of the Y30AY196A+A168F toxoid mixed with Montanide™ ISA 61VG adjuvant (1:1). All of the adjuvanted mixtures were given subcutaneously (s.c.) as 6×0.5 ml doses. Three weeks later the lambs were given a second dose of the adjuvanted protein. Blood samples were taken at the start of the study and at weeks 3, 7 and 12. Lamb immunisations were carried out by Orygen Antibodies Ltd, Penicuik, Scotland).

### Competition ELISA Assay for Measuring Neutralising Antibodies

A competitive ELISA to measure neutralising antibodies was carried out using a Monoscreen ELISA kit (BioX Diagnostics, BIO K 222/2), according to the manufacturers' instructions. Absorbance was read at 450 nm and inhibition calculated using the following formula:

$$\% inhibition sample = [ ( {OD}_{450 n m} negative sera - OD sample ) / {OD}_{450 n m} negative sera ] \times 100$$

$$\% inhibition positive = [ ( {OD}_{450 n m} negative sera - OD positive sera ) / {OD}_{450 n m} negative sera ] \times 100$$

The test was validated only if the OD negative—OD positive was greater than 0.7 and inhibition of the positive control was greater than 30%.

### Neutralisation of Toxicity Towards Cell Cultures

CHO cells expressing Human MAL were seeded at 3×104 cells per well of a 96 well plate and left to settle overnight. Rabbit sera from neat and a standard epsilon antitoxin (National Institute of Biological Standards and Control) from 20 IU/ml were diluted in a series of doubling dilutions in DPBS and incubated with an equal volume of trypsin activated wild type epsilon toxin (5×CT₅₀) for an hour at room temperature. The CHO hMAL cells were washed twice with serum free DMEM/F12 and the toxin: antibody/standard antitoxin mix was then added, along with DPBS only and toxin only controls (5×CT₅₀). Following incubation for 3 hours at 37 °C in a humidified atmosphere, the media was replaced with 100 μls of fresh serum free DMEM/F12 and 10 μl of WST-1 cell proliferation reagent (Abcam). The absorbance at 420 nm was read after incubation for 1 hour at 37 °C in a humidified atmosphere.

### Toxicity in Mice

Groups of six female BALB/c mice were challenged by the intraperitoneal (i.p.) or subcutaneous (s.c.) route with either un-activated or trypsin-activated protein 100 μl volumes. The experiments were was terminated at 24 hours post i.p. challenge or 7 days post s.c. challenge. The studies were performed with the approval of the on-site animal ethics committee. At intervals the animals were assessed for neurological symptoms, changes in appearance or changes in behaviour according to a pre-determined scoring matrix. Animals with a combined score of 5 or more were culled.

### Results

### Mutagenesis of Residues Flanking the β-Octyl-Glucoside Binding Site

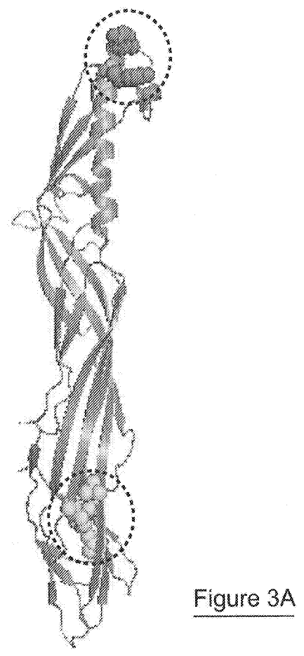

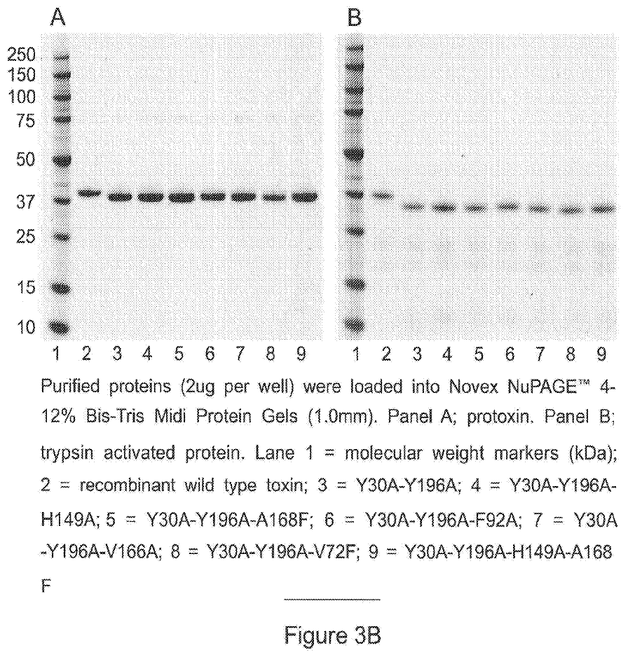

In a previous study the inventors identified a glycan (β-octyl-glucoside) binding site in domain III of Etx and suggested that this site may be a second receptor binding site (Bokori-Brown (2013)). In this study, 5 residues flanking this site were identified for mutagenesis (V72, F92, H149, V166, A168; FIG. 3a) to evaluate their role in toxicity. Using a plasmid which encoded the Y30A-Y196A variant form of Etx, additional mutations in V72, F92, H149, V166 and A168 were introduced. These residues were mutated to alanine (H149, F92, V166) or phenylalanine (A168, V72) and the his-tagged proteins encoded by the mutated genes were expressed in E. coli and purified. Also expressed and purified was the Y30A-Y196A variant form of Etx and a mutant which contained both H149A and A168A in addition to Y30A-Y196A. The authenticity of the proteins was verified in two ways. First, the genes encoding the mutant genes were sequenced to validate the presence of the expected mutation. Secondly, the purified proteins were analysed by mass spectrometry to confirm that the experimentally determined mass matched the expected molecular mass of the protein. For studies in mice, rabbits or sheep the proteins had endotoxin levels below 1 endotoxin units (EU)/ml for mice and rabbits, and below 40 (EU)/ml for sheep.

### Thermostability of Proteins

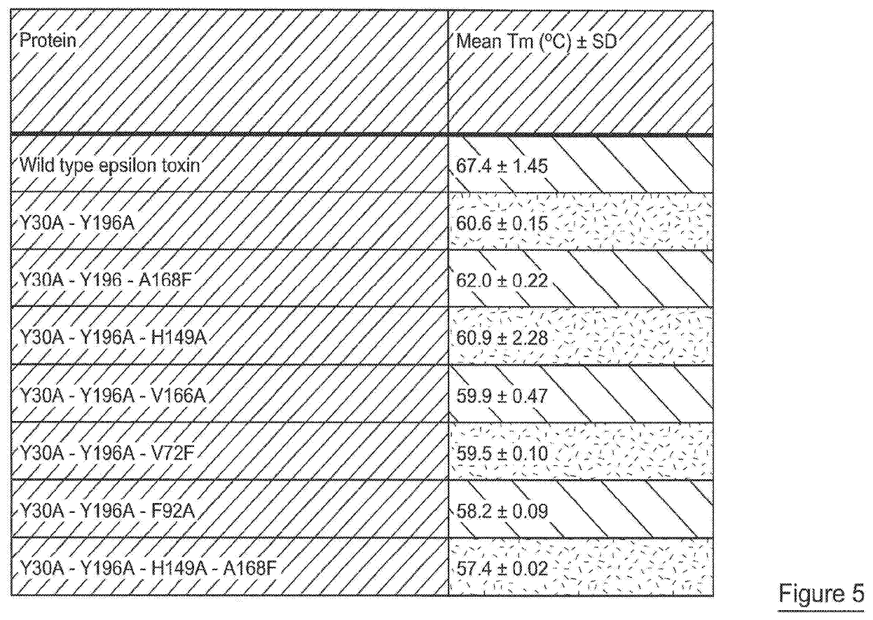

The structural stability of the wild type Epsilon toxin, the Y30A-Y196A variant protein and the 4 mutants of Y30A-Y196A were assessed using a thermostability assay. This revealed that the melting temperature (Tm in °C) of the Y30A-Y196A was lower than that of wild type toxin. However, the introduction of additional mutations (V72, F92, H149, V166 and A168) into the Y30A-Y196A variant protein only resulted in small changes in the thermostability of the proteins (FIG. 5) indicating that these substitutions did not destabilise the tertiary structures of the protein. The Y30A-Y196A-H149A-A168F has the lowest melting temperature, indicating that this was the least stable of the mutants tested.

### Toxicity of the Variant Proteins in Cell Culture

The trypsin activated purified Etx proteins were tested for toxicity towards MDCK.2 cells, CHO cells and CHO cells expressing hMAL, sMAL or dMAL (Table 5). As previously found, the Y30AY196A mutations resulted in over 400-fold reduction in toxicity towards MDCK cells. However, this mutant showed 57-fold increased toxicity towards CHO cells expressing hMAL, 12-fold reduction in toxicity towards CHO cells expressing sMAL and 180 fold reduction in toxicity towards CHO cells expressing dMAL, all compared with wild type toxin. The additional introduction of the H149A, A168F, F92A or V72F mutations into the β-octyl-glucoside binding site reduced toxicity towards CHO-cells expressing hMAL.

TABLE 5
TOXICITY OF THE VARIANT ETX PROTEINS TESTED

| | | | Toxicity | Toxicity | Toxicity towards | Toxicity towards | Toxicity towards | |
|---|---|---|---|---|---|---|---|---|
| | Toxicity | | (activated | (non-activated | CHO-cell | CHO-cell | CHO-cell | |
| | towards | | Epsilon toxin) | Epsilon toxin) | expressing human | expressing sheep | expressing dog | |
| Mutant | MDCK cells | | in mice (i.p.) | in mice (s.c.) | MAL | MAL | MAL | |
| Wild type toxin | 3.47 | nM | 20 ng-200 ng | ND | 12.7 nM ± 0.68 nM | 1.6 nM ± 0.5 nM | 1.7 | nM |
| Y30AY196A | 1.49 | μM | 2 μg-20 μg | ND | 5.5 nM ± 0.43 nM | 13.75 nM ± 0.98 nM | 305 | nM |
| Y30AY196A + H149A | >3 | μM | 2 μg-20 μg | ND | >3 μM | >2 μM | TBD | |
| Y30AY196A + A168F | >3 | μM | >20 μg | >200 μg | >3 μM | >3.0 μM | 1.5 | μM |
| Y30AY196A + F92A | 3 | μM | ND | ND | >3 μM | 52.9 nM ± 17.0 nM | TBD | |
| Y30AY196A + V166A | <3 | μM | ND | ND | 14.6 nM ± 1.4 nM | 76.3 nM ± 11.0 nM | TBD | |
| Y30AY196A + V72F | <3 | μM | <2 μg | ND | >3 μM | TBD | TBD | |
| Y30AY196A + H149A + | >3 | μM | >20 μg | ND | TBD | >3 μM | 788 | nM |
| A168F | | | | | | | | |

### Toxicity of the Variant Proteins in Mice

Variant proteins that had reduced toxicity in CHO-hMAL cell cultures (Y30A-Y196A-H149A, Y30A-Y196A-A168F and Y30A-Y196A-H149A-A168F) were tested for toxicity in mice (Table 5). When given by the i.p. route the MLD dose of trypsin-activated Y30A-Y196A-H149A was between 2 μg and 20 μg whereas the MLD dose of trypsin-activated Y30A-Y196A-A168F or Y30A-Y196A-H149A-A168F was above the highest dose tested (20 μg). The Y30A-Y196A-A168F protein was selected for further testing for toxicity by the s.c. route, either before or after trypsin activation. The MLD doses of the trypsin activated protein was between 20 μg and 200 μg, whereas the non-activated protein was not toxic at the doses tested.

### Antibody Responses to the Variant Proteins

Groups of 3 rabbits were immunised with the Y30A-Y196A, Y30A-Y196A-A168F or Y30A-Y196A-H149A-A168F genetic toxoids given with Freund's incomplete adjuvant. This work was carried out by Cambridge Research Biochemicals (Cleveland, UK). One week after the fourth immunising dose, sera was tested for antibodies able to displace a neutralising monoclonal antibody against epsilon toxin, which indicated the presence of neutralising antibodies in the sera. For comparison we included WHO International Standard Epsilon toxin antitoxin, diluted to 5 IU/ml. The results indicated that all of the rabbits had developed antibodies which reacted with wild type epsilon toxin when tested using competition ELISA. The titres were broadly similar in the sera from individual rabbits in each immunisation group, and we therefore pooled the sera for subsequent tests.

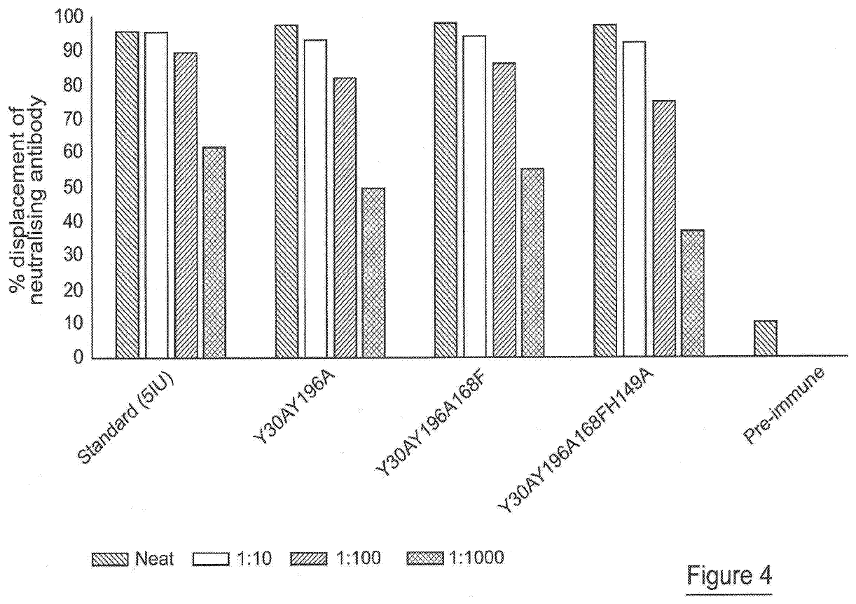

We found that undiluted sera or sera diluted 10-fold in PBS were similar to each other and to the International Standard Epsilon toxin antitoxin in their abilities to displace the neutralising antibody. When diluted 100-fold, the sera raised against Y30A-Y196A, Y30A-Y196A-A168F and Y30A-Y196A-H149A-A168F were all more potent in displacing the neutralising antibody than the International Standard epsilon toxin antitoxin (FIG. 4).

### Immunisation of Lambs

We immunised groups of 5 or 6 lambs with 2 doses of Y30A-Y196A-A168F prototoxin given with either Montanide™ ISA 61VG or alhydrogel adjuvant (Table 6). One additional group of lambs had pre-existing antibodies against epsilon toxin at the start of the study, detected using Western blotting, and these lambs were immunised with 2 doses of Y30A-Y196A-A168F protein with Montanide™ ISA 61VG adjuvant. The sera from these animals and control sera from lambs which had not been immunised were tested for the presence of antibody able to neutralise epsilon toxin using two different assays. First, we used a competition ELISA to measure the ability of the lamb sera to displace a neutralising monoclonal antibody. We also tested the ability of the sera to neutralise toxicity of epsilon toxin towards CHO-hMAL cells. In both of these assays we included dilutions of standardised sera containing a known concentration of neutralising antibody, expressed as international units (IU/ml).

We did not detect antibody in the control lambs using either assay. Using the competition ELISA we detected low levels of antibody in the group immunised with toxoid and alhydrogel adjuvant, but using the CHO-hMAL assay we could not detect any neutralising antibody. In contrast, lambs immunised with toxoid in Montanide™ ISA 61VG adjuvant developed high levels of neutralising antibody in both assays, in excess of 64 IU/ml in the CHO-hMAL assay and 200 IU/ml in the competition ELISA assay (Table 6). Lambs that were immunised with toxoid in Montanide™ ISA 61VG adjuvant but had pre-existing antibodies against epsilon toxin developed lower levels of neutralising antibodies after immunisation relative to lambs immunised with toxoid in Montanide™ ISA 61VG adjuvant that had no pre-existing antibodies against epsilon toxin.

TABLE 6
NEUTRALISING ANTIBODY IN LAMBS
IMMUNISED WITH Y30A-Y196A-A168F

| | Neu- | Neu- | Neu- |
|---|---|---|---|
| | tralising | tralising | tralising |
| | antibody | antibody | antibody |
| | (IU/ml) | (IU/ml) | (IU/ml) |
| | measured | measured | measured |
| | using | using | using |
| | competition | CHO- | CHO- |
| | ELISA | hMal | sMal |
| Treatment Group | assay | cells | cells |
| Control | 0 | 0 | 0 |
| Y30A-Y196A-A168F + | 200 IU/ml | >64 IU/ml | >160 IU/ml |
| Montanide ™ ISA | | | |
| 61VG adjuvant | | | |
| Y30A-Y196A-A168F + | 12.5 IU/ml | 0 | 0 |
| alhydrogel adjuvant | | | |
| Y30A-Y196A-A168F + | 25 IU/ml | >8 IU/ml | >40 IU/ml |
| Montanide ™ ISA | | | |
| 61VG adjuvant | | | |
| (lambs with pre- | | | |
| existing antibodies | | | |
| to epsilon toxin) | | | |

hMAL = human MAL
sMAL = sheep MAL

### DISCUSSION

In a previous study, the inventors investigated the potential of a site-directed mutant of Etx with mutations in the putative receptor binding domain (domain 1) and showed that a combination of Y30A and Y196A mutations markedly reduced the ability of the toxin to bind to and kill MDCK cells. The inventors also previously showed that Y30A-Y196A had reduced toxicity in mice, suggesting that Y30A-Y196A mutant could form the basis of an improved recombinant vaccine against enterotoxemia. Polyclonal antibody raised against Y30A-Y196A provided protection against wild type toxin in an in vitro neutralisation assay.

The previous study however used the MDCK cell line to measure cytotoxicity, and subsequently it has been shown that CHO cell expressing MAL are also highly sensitive to the toxin. The possibility that MAL is a receptor for the toxin is supported by the finding that MAL knockout mice become resistant to the effects of Etx. In the present study forming the basis of this application, it was found that the Y30A-Y196A mutant is only marginally less toxic towards CHO cells expressing sheep MAL, and is more toxic to CHO cells expressing human MAL compared to wild type Etx. However, in CHO cells expressing dog MAL the mutant is markedly less toxic. This finding suggests that MAL from different species interacts differently with Etx, indicating that in future studies MDCK cells as well as CHO cells expressing MAL should both be used in parallel.

The inventors introduced additional mutations to reduce the toxicity of Y30A-Y196A towards CHO cells expressing MAL. The mutations were introduced into a region in domain 3 that has been implicated in sugar binding. A number of these mutants showed reduced toxicity in CHO-hMAL cell cultures and also towards MDCK cells and in mice. The data confirm the role of this region in toxicity. The inventors were able to produce all of these proteins and on the basis of thermostability measurement they did not appear to show major changes in stability, suggesting that the conformation of the proteins was broadly similar to that of the wild type epsilon toxin. The Y30AY196A+A168F mutant was selected for testing in sheep because it induced robust antibody responses in rabbits.

Livestock vaccines containing aluminium hydroxide or saponin as adjuvants often induce short-lived antibody responses (Khorasani et al., (2016) Iranian journal of veterinary research 17: 8-12). This necessitates boosting at intervals, sometimes as short as 4 months apart. Montanide™ ISA 61 VG is a new ready-to-use mineral oil-based adjuvant for use in livestock which offers the potential to induce high level and long-lasting responses in animals (Khorasani et al., (2016)). The finding that the use of Montanide™ ISA 61VG adjuvant resulted in the induction of better antibody responses compared to the use of an alhydrogel adjuvant is similar to previous finding with a foot and mouth disease vaccine (Khorasani et al., (2016)). A previous report has also shown that ISA 61VG is superior to ISA 201 VG (water-in-oil) adjuvant or Montanide™ Gel 01 (aqueous polymer) adjuvant for the induction of antibody responses (Petermann et al., (2017) Exp Appl Acarol. 72:303-315). No evidence of local side effects after using Montanide™ ISA 61VG adjuvanted protein in lambs was seen, although others have reported evidence of local side effects using this adjuvant (Petermann et al., (2017) Exp Appl Acarol. 72:303-315). For licensing of epsilon-toxoid vaccine in Europe, compliance with the European Pharmacopoeia (Ph. Eur.) monograph on Clostridium perfringens vaccines for veterinary use (0363) would be required. The toxoid generated shows residual toxicity which is lower than that required, and the levels of neutralising antibody that we have achieved using Montanide™ ISA 61VG adjuvant is at least 10 times the threshold required of 5 IU/ml.

The finding here that lambs with pre-existing antibodies against epsilon toxin responded less well to vaccination is in line with studies in other species. For example, antibodies inhibit responses to a broad range of vaccines (Voysey et al., (2017) JAMA paediatrics 171:637-46; Edwards et al., (2015) Vaccine 33:6469-72; Idoko et al., (2014) Vaccine 32:4220-7; Zarnitsyna et al., (2016) PLoS Pathog. 12:e1005692). These pre-existing antibodies might result in clearance of the antigen or the formation of antigen-antibody complexes limiting B cell activation or by physically masking the epitope from B cells (Zarnitsyna et al., (2016)).

The vaccine devised here would be used in livestock susceptible to enterotoxaemia caused by C. perfringens epsilon toxin. It would have a number of advantages over existing vaccines because it does not require de-toxification before use. The purity of the antigen and use of an adjuvant such as Montanide™ ISA 61VG should promote long term immunity, reducing or eliminating the need for booster immunisations. In addition, it could serve as a protein carrier for polysaccharides which induce protective antibodies against other diseases of livestock (Petermann et al., (2017) Exp Appl Acarol. 72:303-315.; Voysey et al., (2017); Edwards et al., (2015) Idoko et al., (2014); Zarnitsyna et al., (2016); Byrd et al., (1992) Veterinary immunology and immunopathology 34:307-24). A glycoconjugate would promote T-cell responses to the polysaccharide moiety (Avci et al., (2011) Nature medicine 17:1602-9) with increases in the magnitude of the antibody response and the induction of memory responses to the polysaccharide (Avci (2013) Current Topics in Medicinal Chemistry 13:2535-40); Pace (2013) Expert opinion on biological therapy 13:11-33). In addition, the linking of the polysaccharide to the epsilon toxin carrier would allow the vaccine to be used in young animals (Pace (2013)). The Y30AY196A+A168F protein could be chemically coupled to polysaccharides or it could be further modified to serve as an acceptor for recombinant glycoconjugates generated by exploiting the naturally occurring glycosylation systems in bacteria (Valguarnera et al., (2016) J Mol Biol. 428:3206-20; Cuccui et al., (2015) The Journal of pharmacy and pharmacology 67:338-50.

Finally, the Y30AY196A+A168F protein could in the future be exploited as a vaccine for use in humans. C. perfringens epsilon toxin has been suggested to be a potential biothreat agent (Greenfield et al., (2002) Am J Med Sci. 323:326-40;

Berger et al., (2016) Disaster and military medicine 2:7), and vaccination of at risk individuals would protect them from disease. In addition, epsilon toxin has recently been implicated as playing a role in the development of multiple sclerosis (Rumah et al., (2013) PLoS One 8:e76359; Rumah et al., (2015) PLoS Pathog. 11:e1004896; Linden et al., (2015) mBio. 6). Should this link be established then vaccination to protect against the toxin could be a potential preventative or therapeutic option.

### Longevity of the Protective Antibody Response

Lambs were reared without vaccination against C. perfringens epsilon toxin and after 12 months one group of 5 lambs received 200 μg of Y30AY196A+A168F toxoid adjuvanted (1:1) with Montanide ISA 61VG (Seppic, Paris, France) given subcutaneously (s.c.) as 6×0.5 ml doses. Three weeks later the lambs were given a second dose of the adjuvanted protein. Blood samples were taken at the start of the study and at intervals up to 12 months post immunisation. A competitive ELISA to measure neutralising antibodies was carried out using a Monoscreen ELISA kit (BioX Diagnostics, BIO K 222/2), according to the manufacturers' instructions. We included dilutions of standardised sera containing a known concentration of neutralising antibody, expressed as international units (IU/ml) to enable us to calculate the antibody level expressed as IU/I of neutralising antibody. The results are shown below in Table 7.

TABLE 7
Neutralising antibody in the sera after immunisation
with Y30AY196A + A168F adjuvanted with
Montanide ISA 61VG at week 0 and week 3

| | Weeks post first immunisation | | | | | | |
|---|---|---|---|---|---|---|---|
| | 3 | 7 | 13 | 16 | 25 | 38 | 52 |
| neutralising | 107 | 311 | 231 | 207 | 146 | 85 | 68 |
| antibody (IU/ml) | | | | | | | |

The neutralising antibody titres found in sheep dosed with our toxoid with Montanide ISA 61VG adjuvant are well in excess of the reported minimum protective titres in sheep (0.1-0.3 IU/ml (de la Rosa et al. 1997 (J Anim Sci 75(9): 2328-2334); Uzal and Kelly 1998 (Veterinary Record 142(26): 722-725); or in goats (1 IU/ml (Uzal, Bodero et al. 1998 (Vet Rec 143(17): 472-474), Uzal and Kelly 1998 (Veterinary Record 142(26): 722-725)) and were still above this threshold one year after immunisation. Additionally, neutralising antibody levels were in excess of the protective titres after one dose of our vaccine (i.e. at week 3) indicating that a single dose vaccine for use in livestock is achievable.

### NMO, ON and TM Patient Sample Testing

Table 8 below shows Western blot data from samples taken from subjects with NMO, almost all of whom initially presented with ON and/or TM. 15 of the 30 samples (50%) show very strongly positive (3), strongly positive (2), positive (8) or weakly positive (2) reactivity towards Etx compared with controls, of which 2/25 (8%) were positive.

All subjects tested positive for AQP-4 antibodies and 11 of the 15 subjects who tested positive for Etx initially showed features of Transverse Myelitis™ and/or optic neuritis (ON), examples of neuromyelitis optica spectrum disorder (NMOSD).

TABLE 8
Western Blot Data

| | | Age & | Control |
|---|---|---|---|
| | | gender | Reactivity |
| | Reactivity | Matched | towards Etx |
| NMO/ON/ | towards Etx | Control | by Western |
| TM patient | by Western | (from MS | Blot (from MS |
| sample ID | Blot | study) | study) |
| 1 | ++ | | |
| 2 | − | C1 | − |
| 3 | + | B8 | − |
| 4 | + | A1 | + |
| 5 | + | B12 | − |
| 6 | − | A6/A7 | − |
| 7 | − | D6 | − |
| 8 | − | B7 | − |
| 9 | − | C1 | − |
| 10 | − | A6 | − |
| 11 | − | C1 | − |
| 12 | − | B9/E3/E2 | − |
| 13 | + | A1 | − |
| | (very weak) | | |
| 14 | + | B7 | − |
| 15 | + | D6/D2/ | − |
| | | B2/D11 | |
| 16 | − | | |
| 17 | + | C2 | − |
| 18 | − | F2 | − |
| 19 | +++ | B1 | − |
| 20 | ++ | B1 | − |
| 21 | − | | |
| 22 | + | B1 | − |
| 23 | − | Exeter 19 | − |
| 24 | − | | |
| 25 | − | B11 | + |
| 26 | + | C9 | − |
| | (very weak) | | |
| 27 | +++ | C9/C10 | − |
| 28 | +++ | | |
| 29 | − | B3 | − |
| 30 | + | B5 | − |
| TOTAL +ve | 15/30 (50%) | | 2/25 (8%) |

Controls in bold italics-Gender and age matched +/− 2 years

Treatment of Human Red Blood Cells (RBCs) with Epsilon Toxin

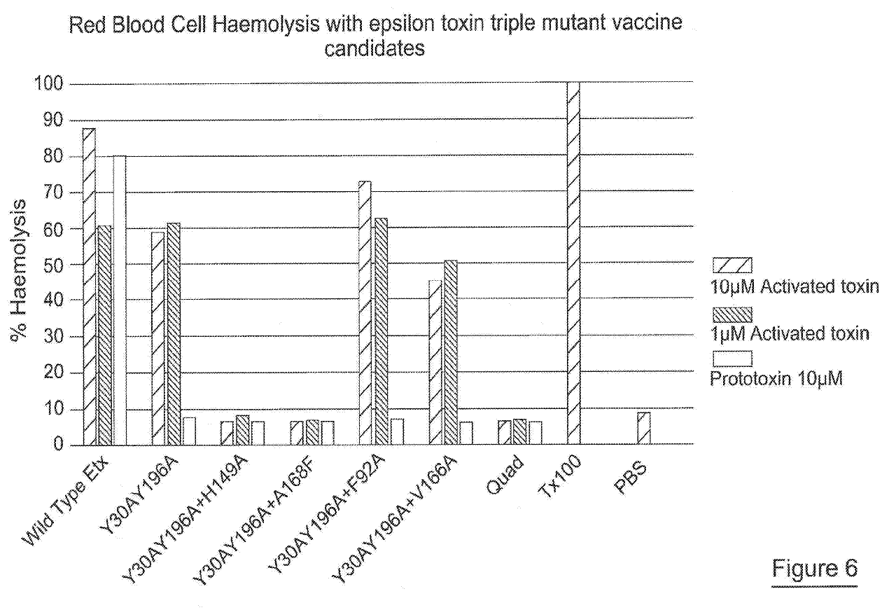

Human red blood cells were tested for haemolysis when exposed to wild type Etx; Y30AY196A; Y30AY196A+H149A; Y30AY196A+A168F; Y30AY196A+F92A; Y30AY196A+V166A, and a Quad: Y30AY196+A168F+H149A. The prototoxin at 10 μM was tested and trypsin activated toxin at 10 μM and 1 μM with 3.3% RBCs. Each toxin was tested in triplicate at each dose. Results are expressed with respect to 1% Tx100 control (resulting in 100% haemolysis); negative control=PBS. The results are shown in FIG. 6 and illustrate that Y30AY196A+H149A, Y30AY196A+A168F and Y30AY196+A168F+H149A are not haemolytic even when trypsin activated.

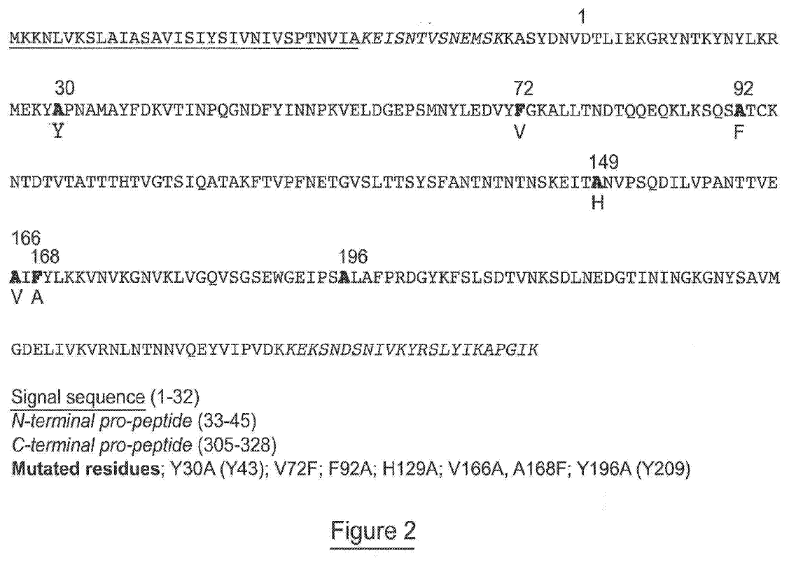

SEQUENCES (MUTATION POSITIONS SHOWN IN BOLD)
SEQ ID NO: 1
VYVGKALLTNDTQQEQKLKSQSFTCK
SEQ ID NO: 2
THNVPSQDILVPANTTVEVIAYLK
SEQ ID NO: 3
DELIVKVRNLNTNNVQEYVIPVDKKEKSNDSNIVKYRSLYIKAPGIK
Y30A mutation
SEQ ID NO: 4
RMEKYXPNAM
Y196A mutation
SEQ ID NO: 5
GEIPSXLAFP
H149A mutation
SEQ ID NO: 6
SKEITXNVPS
V72F mutation
SEQ ID NO: 7
LEDVYXGKAL
F92A mutation
SEQ ID NO: 8
LKSQSXTCKN
V166A mutation
SEQ ID NO: 9
NTTVEXIAYL
A168F mutation
SEQ ID NO: 10
TVEVIXYLKK
full length wild-type native epsilon toxin
SEQ ID NO: 11
MKKNLVKSLAIASAVISIYSIVNIVSPTNVIAKEISNTVSNEMSKKASYD
NVDTLIEKGRYNTKYNYLKRMEKYYPNAMAYFDKVTINPQGNDFYINNPK
VELDGEPSMNYLEDVYVGKALLTNDTQQEQKLKSQSFTCKNTDTVTATTT
HTVGTSIQATAKFTVPFNETGVSLTTSYSFANTNTNINSKEITHNVPSQD
ILVPANTTVEVIAYLKKVNVKGNVKLVGQVSGSEWGEIPSYLAFPRDGYK
FSLSDTVNKSDLNEDGTININGKGNYSAVMGDELIVKVRNLNTNNVQEYV
IPVDKKEKSNDSNIVKYRSLSIKAPGIK
sequence used to obtain crystal structure
(PDB ID:1YUJ)
SEQ ID NO: 12
KEISNTVSNEMSKKASYDNVDTLIEKGRYNTKYNYLKRMEKYYPNAMAYF
DKVTINPQGNDFYINNPKVELDGEPSMNYLEDVYVGKALLINDTQQEQKL
KSQSFTCKNTDTVTATTTHTVGTSIQATAKFTVPFNETGVSLTTSYSFAN
TNTNTNSKEITHNVPSQDILVPANTTVEVIAYLKKVNVKGNVKLVGQVSG
SEWGEIPSYLAFPRDGYKFSLSDTVNKSDLNEDGTININGKGNYSAVMGD
ELIVKVRNLNTNNVQEYVIPVDKKEKSNDSNIVKYRSLSIKAPGIK
trypsin activated wild-type recombinant
epsilon toxin
SEQ ID NO: 13
MGKASYDNVDTLIEKGRYNTKYNYLKRMEKYYPNAMAYFDKVTINPQGND
FYINNPKVELDGEPSMNYLEDVYVGKALLTNDTQQEQKLKSQSFTCKNTD
TVTATTTHTVGTSIQATAKFTVPFNETGVSLTTSYSFANTNTNTNSKEIT
HNVPSQDILVPANTTVEVIAYLKKVNVKGNVKLVGQVSGSEWGEIPSYLA
FPRDGYKFSLSDTVNKSDLNEDGTININGKGNYSAVMGDELIVKVRNLNT
NNVQEYVIPVDKK
trypsin activated recombinant epsilon toxin
with H149A mutation
SEQ ID NO: 14
MGKASYDNVDTLIEKGRYNTKYNYLKRMEKYYPNAMAYFDKVTINPQGND
FYINNPKVELDGEPSMNYLEDVYVGKALLTNDTQQEQKLKSQSFTCKNTD
TVTATTTHTVGTSIQATAKFTVPFNETGVSLTTSYSFANTNTNTNSKEIT
ANVPSQDILVPANTTVEVIAYLKKVNVKGNVKLVGQVSGSEWGEIPSYLA
FPRDGYKFSLSDTVNKSDLNEDGTININGKGNYSAVMGDELIVKVRNLNT
NNVQEYVIPVDKK
full length recombinant epsilon toxin
SEQ ID NO: 15
MKYLLPTAAAGLLLLAAQPAMAMGKASYDNVDTLIEKGRYNTKYNYLKRM
EKYYPNAMAYFDKVTINPQGNDFYINNPKVELDGEPSMNYLEDVYVGKAL
LTNDTQQEQKLKSQSFTCKNTDTVTATTTHTVGTSIQATAKFTVPFNETG
VSLTTSYSFANTNTNTNSKEITHNVPSQDILVPANTTVEVIAYLKKVNVK
GNVKLVGQVSGSEWGEIPSYLAFPRDGYKFSLSDTVNKSDLNEDGTININ
GKGNYSAVMGDELIVKVRNLNTNNVQEYVIPVDKKEKSNDSNIVKYRSLY
IKAPGIKLEHHHHHH
trypsin activated recombinant epsilon toxin
(Y30A + Y196A)
SEQ ID NO: 16
MGKASYDNVDTLIEKGRYNTKYNYLKRMEKYAPNAMAYFDKVTINPQGND
FYINNPKVELDGEPSMNYLEDVYVGKALLTNDTQQEQKLKSQSFTCKNID
TVTATTTHTVGTSIQATAKFTVPFNETGVSLTTSYSFANTNTNTNSKEIT
HNVPSQDILVPANTTVEVIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + H149A)
SEQ ID NO: 17
MGKASYDNVDTLIEKGRYNTKYNYLKRMEKYAPNAMAYFDKVTINPQGND
FYINNPKVELDGEPSMNYLEDVYVGKALLTNDTQQEQKLKSQSFTCKNTD
TVTATTTHTVGTSIQATAKFTVPFNETGVSLTTSYSFANTNTNTNSKEIT
ANVPSQDILVPANTTVEVIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + V72F)
SEQ ID NO: 18
MGKASYDNVDTLIEKGRYNTKYNYLKRMEKYAPNAMAYFDKVTINPQGND
FYINNPKVELDGEPSMNYLEDVYFGKALLTNDTQQEQKLKSQSFTCKNTD
TVTATTTHTVGTSIQATAKFTVPFNETGVSLTTSYSFANTNTNTNSKEIT
HNVPSQDILVPANTTVEVIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + F92A)
SEQ ID NO: 19
MGKASYDNVDTLIEKGRYNTKYNYLKRMEKYAPNAMAYFDKVTINPQGND
FYINNPKVELDGEPSMNYLEDVYVGKALLTNDTQQEQKLKSQSATCKNTD
TVTATTTHTVGTSIQATAKFTVPFNETGVSLTTSYSFANTNTNTNSKEIT
HNVPSQDILVPANTTVEVIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + V166A)
SEQ ID NO: 20
MGKASYDNVDTLIEKGRYNTKYNYLKRMEKYAPNAMAYFDKVTINPQGND
FYINNPKVELDGEPSMNYLEDVYVGKALLTNDTQQEQKLKSQSFTCKNTD
TVTATTTHTVGTSIQATAKFTVPFNETGVSLTTSYSFANTNTNTNSKEIT
HNVPSQDILVPANTTVEAIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + A168F)
SEQ ID NO: 21
MGKASYDNVDTLIEKGRYNTKYNYLKRMEKYAPNAMAYFDKVTINPQGND
FYINNPKVELDGEPSMNYLEDVYVGKALLTNDTQQEQKLKSQSFTCKNTD
TVTATTTHTVGTSIQATAKFTVPFNETGVSLTTSYSFANTNTNTNSKEIT
HNVPSQDILVPANTTVEVIFYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + H149A + V72F)
SEQ ID NO: 22
MGKASYDNVDTLIEKGRYNTKYNYLKRMEKYAPNAMAYFDKVTINPQGND
FYINNPKVELDGEPSMNYLEDVYFGKALLTNDTQQEQKLKSQSFTCKNTD
TVTATTTHTVGTSIQATAKFTVPFNETGVSLTTSYSFANTNTNTNSKEIT
ANVPSQDILVPANTTVEVIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + H149A + F92A)
SEQ ID NO: 23
MGKASYDNVDTLIEKGRYNTKYNYLKRMEKYAPNAMAYFDKVTINPQGND
FYINNPKVELDGEPSMNYLEDVYVGKALLTNDTQQEQKLKSQSATCKNTD
TVTATTTHTVGTSIQATAKFTVPFNETGVSLTTSYSFANTNTNTNSKEIT
ANVPSQDILVPANTTVEVIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + H149A + V166A)
SEQ ID NO: 24
MGKASYDNVDTLIEKGRYNTKYNYLKRMEKYAPNAMAYFDKVTINPQGND
FYINNPKVELDGEPSMNYLEDVYVGKALLTNDTQQEQKLKSQSFTCKNTD
TVTATTTHTVGTSIQATAKFTVPFNETGVSLTTSYSFANTNTNTNSKEIT
ANVPSQDILVPANTTVEAIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + H149A + A168F)
SEQ ID NO: 25
MGKASYDNVDTLIEKGRYNTKYNYLKRMEKYAPNAMAYFDKVTINPQGND
FYINNPKVELDGEPSMNYLEDVYVGKALLTNDTQQEQKLKSQSFTCKNTD
TVTATTTHTVGTSIQATAKFTVPFNETGVSLTTSYSFANTNTNTNSKEIT
ANVPSQDILVPANTTVEVIFYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + V72F + F92A)
SEQ ID NO: 26
MGKASYDNVDTLIEKGRYNTKYNYLKRMEKYAPNAMAYFDKVTINPQGND
FYINNPKVELDGEPSMNYLEDVYFGKALLTNDTQQEQKLKSQSATCKNTD
TVTATTTHTVGTSIQATAKFTVPFNETGVSLTTSYSFANTNTNTNSKEIT
HNVPSQDILVPANTTVEVIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + V72F + V166A)
SEQ ID NO: 27
MGKASYDNVDTLIEKGRYNTKYNYLKRMEKYAPNAMAYFDKVTINPQGND
FYINNPKVELDGEPSMNYLEDVYFGKALLTNDTQQEQKLKSQSFTCKNTD
TVTATTTHTVGTSIQATAKFTVPFNETGVSLTTSYSFANTNTNTNSKEIT
HNVPSQDILVPANTTVEAIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + V72F + A168F)
SEQ ID NO: 28
MGKASYDNVDTLIEKGRYNTKYNYLKRMEKYAPNAMAYFDKVTINPQGND
FYINNPKVELDGEPSMNYLEDVYFGKALLTNDTQQEQKLKSQSFTCKNTD
TVTATTTHTVGTSIQATAKFTVPFNETGVSLTTSYSFANTNTNTNSKEIT
HNVPSQDILVPANTTVEVIFYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + F92A + V166A)
SEQ ID NO: 29
MGKASYDNVDTLIEKGRYNTKYNYLKRMEKYAPNAMAYFDKVTINPQGND
FYINNPKVELDGEPSMNYLEDVYVGKALLTNDTQQEQKLKSQSATCKNTD
TVTATTTHTVGTSIQATAKFTVPFNETGVSLTTSYSFANTNTNTNSKEIT
HNVPSQDILVPANTTVEAIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + F92A + A168F)
SEQ ID NO: 30
MGKASYDNVDTLIEKGRYNTKYNYLKRMEKYAPNAMAYFDKVTINPQGND
FYINNPKVELDGEPSMNYLEDVYVGKALLTNDTQQEQKLKSQSATCKNTD
TVTATTTHTVGTSIQATAKFTVPFNETGVSLTTSYSFANTNTNTNSKEIT
HNVPSQDILVPANTTVEVIFYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + V166A + A168F)
SEQ ID NO: 31
MGKASYDNVDTLIEKGRYNTKYNYLKRMEKYAPNAMAYFDKVTINPQGND
FYINNPKVELDGEPSMNYLEDVYVGKALLTNDTQQEQKLKSQSFTCKNTD
TVTATTTHTVGTSIQATAKFTVPFNETGVSLTTSYSFANTNTNTNSKEIT
HNVPSQDILVPANTTVRAIFYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + H149A + V72F + F92A)
SEQ ID NO: 32
MGKASYDNVDTLIEKGRYNTKYNYLKRMEKYAPNAMAYFDKVTINPQGND
FYINNPKVELDGEPSMNYLEDVYFGKALLTNDTQQEQKLKSQSATCKNTD
TVTATTTHTVGTSIQATAKFTVPFNETGVSLTTSYSFANTNTNTNSKEIT
ANVPSQDILVPANTTVEVIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + H149A + V72F + V166A)
SEQ ID NO: 33
MGKASYDNVDTLIEKGRYNTKYNYLKRMEKYAPNAMAYFDKVTINPQGND
FYINNPKVELDGEPSMNYLEDVYFGKALLTNDTQQEQKLKSQSFTCKNTD
TVTATTTHTVGTSIQATAKFTVPFNETGVSLTTSYSFANTNTNTNSKEIT
ANVPSQDILVPANTTVEAIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + H149A + V72F + A168F)
SEQ ID NO: 34
MGKASYDNVDTLIEKGRYNTKYNYLKRMEKYAPNAMAYFDKVTINPQGND
FYINNPKVELDGEPSMNYLEDVYFGKALLTNDTQQEQKLKSQSFTCKNTD
TVTATTTHTVGTSIQATAKFTVPFNETGVSLTTSYSFANTNTNTNSKEIT
ANVPSQDILVPANTTVEVIFYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + H149A + F92A + V166A)
SEQ ID NO: 35
MGKASYDNVDTLIEKGRYNTKYNYLKRMEKYAPNAMAYFDKVTINPQGND
FYINNPKVELDGEPSMNYLEDVYVGKALLTNDTQQEQKLKSQSATCKNTD
TVTATTTHTVGTSIQATAKFTVPFNETGVSLTTSYSFANTNTNTNSKEIT
ANVPSQDILVPANTTVEAIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + H149A + F92A + A168F)
SEQ ID NO: 36
MGKASYDNVDTLIEKGRYNTKYNYLKRMEKYAPNAMAYFDKVTINPQGND
FYINNPKVELDGEPSMNYLEDVYVGKALLTNDTQQEQKLKSQSATCKNTD
TVTATTTHTVGTSIQATAKFTVPFNETGVSLTTSYSFANTNTNTNSKEIT
ANVPSQDILVPANTTVEVIFYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + H149A + V166A + A168F)
SEQ ID NO: 37
MGKASYDNVDTLIEKGRYNTKYNYLKRMEKYAPNAMAYFDKVTINPQGND
FYINNPKVELDGEPSMNYLEDVYVGKALLTNDTQQEQKLKSQSFTCKNTD
TVTATTTHTVGTSIQATAKFTVPFNETGVSLTTSYSFANTNTNTNSKEIT
ANVPSQDILVPANTTVEAIFYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + V72F + F92A + V166A)
SEQ ID NO: 38
MGKASYDNVDTLIEKGRYNTKYNYLKRMEKYAPNAMAYFDKVTINPQGND
FYINNPKVELDGEPSMNYLEDVYFGKALLTNDTQQEQKLKSQSATCKNTD
TVTATTTHTVGTSIQATAKFTVPFNETGVSLTTSYSFANTNTNTNSKEIT
HNVPSQDILVPANTTVEAIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + V72F + F92A + A168F)
SEQ ID NO: 39
MGKASYDNVDTLIEKGRYNTKYNYLKRMEKYAPNAMAYFDKVTINPQGND
FYINNPKVELDGEPSMNYLEDVYFGKALLTNDTQQEQKLKSQSATCKNTD
TVTATTTHTVGTSIQATAKFTVPFNETGVSLTTSYSFANTNTNTNSKEIT
HNVPSQDILVPANTTVEVIFYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + V72F + V166A + A168F)
SEQ ID NO: 40
MGKASYDNVDTLIEKGRYNTKYNYLKRMEKYAPNAMAYFDKVTINPQGND
FYINNPKVELDGEPSMNYLEDVYFGKALLTNDTQQEQKLKSQSFTCKNTD
TVTATTTHTVGTSIQATAKFTVPFNETGVSLTTSYSFANTNTNTNSKEIT
HNVPSQDILVPANTTVEAIFYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + F92A + V166A + A168F)
SEQ ID NO: 41
MGKASYDNVDTLIEKGRYNTKYNYLKRMEKYAPNAMAYFDKVTINPQGND
FYINNPKVELDGEPSMNYLEDVYVGKALLTNDTQQEQKLKSQSATCKNTD
TVTATTTHTVGTSIQATAKFTVPFNETGVSLTTSYSFANTNTNTNSKEIT
HNVPSQDILVPANTTVRAIFYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + H149A + V72F + F92A + V166A)
SEQ ID NO: 42
MGKASYDNVDTLIEKGRYNTKYNYLKRMEKYAPNAMAYFDKVTINPQGND
FYINNPKVELDGEPSMNYLEDVYFGKALLTNDTQQEQKLKSQSATCKNTD
TVTATTTHTVGTSIQATAKFTVPFNETGVSLTTSYSFANTNTNTNSKEIT
ANVPSQDILVPANTTVEAIAYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + H149A + V72F + F92A + A168F)
SEQ ID NO: 43
MGKASYDNVDTLIEKGRYNTKYNYLKRMEKYAPNAMAYFDKVTINPQGND
FYINNPKVELDGEPSMNYLEDVYFGKALLTNDTQQEQKLKSQSATCKNTD
TVTATTTHTVGTSIQATAKFTVPFNETGVSLTTSYSFANTNTNTNSKEIT
ANVPSQDILVPANTTVEVIFYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + V72F + F92A + V166A + A168F)
SEQ ID NO: 44
MGKASYDNVDTLIEKGRYNTKYNYLKRMEKYAPNAMAYFDKVTINPQGND
FYINNPKVELDGEPSMNYLEDVYFGKALLTNDTQQEQKLKSQSATCKNTD
TVTATTTHTVGTSIQATAKFTVPFNETGVSLTTSYSFANTNTNTNSKEIT
HNVPSQDILVPANTTVRAIFYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(Y30A + Y196A + H149A + V72F + F92A + V166A + A168F)
SEQ ID NO: 45
MGKASYDNVDTLIEKGRYNTKYNYLKRMEKYAPNAMAYFDKVTINPQGND
FYINNPKVELDGEPSMNYLEDVYFGKALLTNDTQQEQKLKSQSATCKNTD
TVTATTTHTVGTSIQATAKFTVPFNETGVSLTTSYSFANTNTNTNSKEIT
ANVPSQDILVPANTTVEAIFYLKKVNVKGNVKLVGQVSGSEWGEIPSALA
FPRDGYKFSLSDTVNKSDLNEDGTININGKGNYSAVMGDELIVKVRNLNT
NNVQEYVIPVDKK
trypsin activated recombinant epsilon toxin
(V72F)
SEQ ID NO: 46
MGKASYDNVDTLIEKGRYNTKYNYLKRMEKYYPNAMAYFDKVTINPQGND
FYINNPKVELDGEPSMNYLEDVYFGKALLTNDTQQEQKLKSQSFTCKNTD
TVTATTTHTVGTSIQATAKFTVPFNETGVSLTTSYSFANTNTNTNSKEIT
HNVPSQDILVPANTTVEVIAYLKKVNVKGNVKLVGQVSGSEWGEIPSYLA
FPRDGYKFSLSDTVNKSDLNEDGTININGKGNYSAVMGDELIVKVRNLNT
NNVQEYVIPVDKK
trypsin activated recombinant epsilon toxin
(F92A)
SEQ ID NO: 47
MGKASYDNVDTLIEKGRYNTKYNYLKRMEKYYPNAMAYFDKVTINPQGND
FYINNPKVELDGEPSMNYLEDVYVGKALLTNDTQQEQKLKSQSATCKNTD
TVTATTTHTVGTSIQATAKFTVPFNETGVSLTTSYSFANTNTNTNSKEIT
HNVPSQDILVPANTTVEVIAYLKKVNVKGNVKLVGQVSGSEWGEIPSYLA
FPRDGYKFSLSDTVNKSDLNEDGTININGKGNYSAVMGDELIVKVRNLNT
NNVQEYVIPVDKK
trypsin activated type recombinant epsilon
toxin (H149A)
SEQ ID NO: 48
MGKASYDNVDTLIEKGRYNTKYNYLKRMEKYYPNAMAYFDKVTINPQGND
FYINNPKVELDGEPSMNYLEDVYVGKALLTNDTQQEQKLKSQSFTCKNTD
TVTATTTHTVGTSIQATAKFTVPFNETGVSLTTSYSFANTNTNTNSKEIT
ANVPSQDILVPANTTVEVIAYLKKVNVKGNVKLVGQVSGSEWGEIPSYLA
FPRDGYKFSLSDTVNKSDLNEDGTININGKGNYSAVMGDELIVKVRNLNT
NNVQEYVIPVDKK
trypsin activated recombinant epsilon toxin
(V166A)
SEQ ID NO: 49
MGKASYDNVDTLIEKGRYNTKYNYLKRMEKYYPNAMAYFDKVTINPQGND
FYINNPKVELDGEPSMNYLEDVYVGKALLTNDTQQEQKLKSQSFTCKNTD
TVTATTTHTVGTSIQATAKFTVPFNETGVSLTTSYSFANTNTNTNSKEIT
HNVPSQDILVPANTTVEAIAYLKKVNVKGNVKLVGQVSGSEWGEIPSYLA
FPRDGYKFSLSDTVNKSDLNEDGTININGKGNYSAVMGDELIVKVRNLNT
NNVQEYVIPVDKK
trypsin activated recombinant epsilon toxin
(A168F)
SEQ ID NO: 50
MGKASYDNVDTLIEKGRYNTKYNYLKRMEKYYPNAMAYFDKVTINPQGND
FYINNPKVELDGEPSMNYLEDVYVGKALLTNDTQQEQKLKSQSFTCKNTD
TVTATTTHTVGTSIQATAKFTVPFNETGVSLTTSYSFANTNTNTNSKEIT
HNVPSQDILVPANTTVEVIFYLKKVNVKGNVKLVGQVSGSEWGEIPSYLA
FPRDGYKFSLSDTVNKSDLNEDGTININGKGNYSAVMGDELIVKVRNLNT
NNVQEYVIPVDKK
Y43A FORWARD
SEQ ID NO: 51
GAAAGGAAGATATAATACAAAATATAATTACTTAAAGAGAATGGAAAAATATGCGCCTAATGCTATGGCATA
TTTTGATAAGG
Y43A REVERSE
SEQ ID NO: 52
CCTTATCAAAATATGCCATAGCATTAGGCGCATATTTTTCCATTCTCTTTAAGTAATTATATTTTGTATTAT
ATCTTCCTTTC
Y209A FORWARD
SEQ ID NO: 53
GTGAATGGGGAGAGATACCTAGTGCGTTAGCTTTTCCTAGGGATGGTTA
Y209A REVERSE
SEQ ID NO: 54
TAACCATCCCTAGGAAAAGCTAACGCACTAGGTATCTCTCCCCATTCAC
H149A FORWARD
SEQ ID NO: 55
CAAATACAAATACAAATACTAATTCAAAAGAAATTACTGCTAATGTCCCTTCACAAGATATACTA
H149A REVERSE
SEQ ID NO: 56
TAGTATATCTTGTGAAGGGACATTAGCAGTAATTTCTTTTGAATTAGTATTTGTATTTGTATTTG
V72F FORWARD
SEQ ID NO: 57
AGAACCATCAATGAATTATCTTGAAGATGTTTATTTTGGAAAAGCTCTCTTAAC
V72F REVERSE
SEQ ID NO: 58
GTTAAGAGAGCTTTTCCAAAATAAACATCTTCAAGATAATTCATTGATGGTTCT
F92A FORWARD
SEQ ID NO: 59
TCTTAACTAATGATACTCAACAAGAACAAAAATTAAAATCACAATCAGCGACTTGTAAAAATACTGATACAG
TAAC
F92A REVERSE
SEQ ID NO: 60
GTTACTGTATCAGTATTTTTACAAGTCGCTGATTGTGATTTTAATTTTTGTTCTTGTTGAGTATCATTAGTT
AAGA
V166A FORWARD
SEQ ID NO: 61
ATACTAGTACCAGCTAATACTACTGTAGAAGCGATAGCATATTTAAAAAAAGTTAATGTTAAAG
V166A REVERSE
SEQ ID NO: 62
CTTTAACATTAACTTTTTTTAAATATGCTATCGCTTCTACAGTAGTATTAGCTGGTACTAGTAT
A168F FORWARD
SEQ ID NO: 63
GATATACTAGTACCAGCTAATACTACTGTAGAAGTAATATTTTATTTAAAAAAAGTTAATGTTAAAGGAAAT
GTAAAGTTAG
A168F REVERSE
SEQ ID NO: 64
CTAACTTTACATTTCCTTTAACATTAACTTTTTTTAAATAAAATATTACTTCTACAGTAGTATTAGCTGGTA
CTAGTATATC
(FIG. 2 residues 1 to 260 of unmutated
sequence; trypsin activated polypeptide)
SEQ ID NO: 65
KASYDNVDTL IEKGRYNTKY NYLKRMEKYY PNAMAYFDKV TINPQGNDFY
INNPKVELDG EPSMNYLEDV YVGKALLTND TQQEQKLKSQ SFTCKNTDTV
TATTTHTVGT SIQATAKFTV PFNETGVSLT TSYSFANTNT NTNSKEITHN
VPSQDILVPA NTTVEVIAYL KKVNVKGNVK LVGQVSGSEW GEIPSYLAFP
RDGYKFSLSD TVNKSDLNED GTININGKGN YSAVMGDELI VKVRNLNTNN
VQEYVIPVDK
(FIG. 2 signal sequence residues 1 to 32)
SEQ ID NO: 66
MKKNLVKSLAIASAVISIYSIVNIVSPTNVIA
(FIG. 2 N-terminal pro-peptide residues 33
to 45)
SEQ ID NO: 67
KEISNTVSNEMSK
(FIG. 2 C-terminal pro-peptide residues
305 to 328)
SEQ ID NO: 68
KEKSNDSNIVKYRSLYIKAPGIK

1. A method for preventing or treating a demyelinating condition selected from:
enterotoxemia (ET), multiple sclerosis (MS), clinically definite MS (CDMS), clinically isolated syndrome (CIS), neuromyelitis optica spectrum disorder (NMOSD), optic neuritis (ON), neuromyelitis optica (NMO), myelitis, myelitis, transverse myelitis (TM), a disease or condition characterised by the increase or presence of antibodies against aquaporin-4 (AQP-4) and/or astrocyte damage, and acute disseminated encephalomyelitis (ADEM) in a human or animal subject in need, comprising: administering to said subject a composition comprising an effective amount of an agent that directly or indirectly interferes with epsilon toxin (Etx) produced by Clostridium perfringens type B or type D bacterial strain, an Etx-binding receptor, or an interaction of Etx with its binding receptor so as to inhibit or suppress Etx modulated receptor signalling activities.

2. The method of claim 1, wherein said agent is an inhibitor of Etx, such as an antibody or a functional component thereof.

3. The method of claim 1, wherein said agent is an inhibitor or antagonist of an Etx-binding receptor.

4. The method of claim 3, wherein said Etx-binding receptor is myelin and lymphocyte protein (MAL) or the hepatitis A virus cell receptor 1 proteins (HAVCR1).

5. The method of claim 1, wherein said agent is a vaccine against Clostridium perfringens type B or type D bacterial strain, or the epsilon toxin (Etx) produced therefrom.

6. The method according to claim 1, wherein said agent comprises an epsilon toxin (Etx) polypeptide having reduced toxicity to cells expressing Myelin And Lymphocyte (MAL) protein and comprising a modified domain III compared to wild type Etx polypeptide SEQ ID NO:65, wherein said reduced toxicity is relative to SEQ ID NO: 65 and/or SEQ ID NO: 14 and wherein said Etx polypeptide is capable of binding at least one antibody which binds to a sequence represented by SEQ ID NO:65 and/or SEQ ID NO:14.

7. The method of claim 6, wherein said modified domain III is a modification in the glycan (β-octyl-glucoside) binding site of domain III.

8. The method according to claim 6, wherein said modified domain III comprises one or more mutations of the amino acids within the amino acid sequences making up domain III as represented by SEQ ID NOs 1, 2 and 3.

9. The method according to claim 6, comprising one or more of the following: SEQ ID NO: 10, SEQ ID NO: 6, SEQ ID NO: 7, SEQ ID NO: 8 and SEQ ID NO: 9.

10. The method according to claim 6, comprising SEQ ID NO: 4 and/or SEQ ID NO: 5 and/or SEQ ID NO: 6.

11. The method according to claim 6, comprising at least the following sequences:
a. SEQ ID NO: 4 and SEQ ID NO: 5;
and optionally in addition to (i)
b. SEQ ID NO: 6;
and
c. one or more of the following: SEQ ID NO:10, SEQ ID NO:7, SEQ ID NO:8 and SEQ ID NO:9.

12. The method according to claim 6, wherein said reduced toxicity is reduced compared to an Etx polypeptide comprising SEQ ID NO: 4 and SEQ ID NO: 5; or an Etx polypeptide comprising SEQ ID NO: 4, SEQ ID NO: 5 and SEQ ID NO: 6; or compared to a known Etx vaccine or Etx vaccine candidate.

13. The method according to claim 6 having at least 60% sequence identity to any one of SEQ ID NOs 18 to 50 and comprising or consisting of the mutation(s) indicated in Table 3 for the relevant SEQ ID NO.

14. A polynucleotide having a nucleic acid sequence which encodes for an agent or polypeptide according to any preceding claim.

15. A vector comprising a polynucleotide according to claim 14.

16. A cell comprising an agent according to claim 1.

17. A subunit or conjugate vaccine comprising an agent according to claim 1.

18. An affinity reagent which is capable of binding to an agent according to claim 1 and facilitating an immune response in the body of an individual to which the affinity reagent is administered.

19. A method for the preparation of an immunotherapy composition, optionally a vaccine composition, comprising adapting an Etx polypeptide or a vaccine comprising an Etx polypeptide by modifying domain III relative to a wild type Etx polypeptide.

20. An immunotherapy or vaccine composition prepared by a method according to claim 19.

21. An immunotherapy or vaccine composition comprising an agent according to claim 1.

22. An immunotherapy composition, optionally a vaccine composition, according to claim 20, which is a foodstuff for a human or animal.

23. A polypeptide according to claim 6 for use in a method of treating or vaccinating a subject against developing a disease caused by or associated with Clostridium perfringens and/or caused by or associated with (active) epsilon toxin and/or against a demyelinating disease.

24. A polypeptide according to claim 23, wherein the disease is selected from: enterotoxemia (ET), multiple sclerosis (MS), clinically definite MS (CDMS), clinically isolated syndrome (CIS), neuromyelitis optica spectrum disorder (NMOSD), optic neuritis (ON), neuromyelitis optica (NMO), myelitis, myelitis, transverse myelitis (TM), a disease or condition characterised by the increase or presence of antibodies against aquaporin-4 (AQP-4) and/or astrocyte damage, and acute disseminated encephalomyelitis (ADEM).

25. A method of treating a subject having a disease caused by or associated with the presence of Clostridium perfringens and/or caused by or associated with the presence of (active) epsilon toxin and/or against a demyelinating disease, or a method for vaccinating a subject against developing such a disease, the method comprising administering to a subject an agent according to claim 1, wherein the demyelinating condition is selected from: enterotoxemia (ET), multiple sclerosis (MS), clinically definite MS (CDMS), clinically isolated syndrome (CIS), neuromyelitis optica spectrum disorder (NMOSD), optic neuritis (ON), neuromyelitis optica (NMO), myelitis, transverse myelitis (TM), a disease or condition characterised by the increase or presence of antibodies against aquaporin-4 (AQP-4) and/or astrocyte damage, and acute disseminated encephalomyelitis (ADEM).

26. A polypeptide, polynucleotide, vector, cell, affinity reagent, vaccine composition or immunotherapy composition according to claim 20, wherein the subject is a ruminant animal, a horse, a companion animal or a human.

27. Use of MAL cells as a model in the testing for toxicity of epsilon vaccine candidates.

28. A kit comprising an agent according to claim 1.

29. A polypeptide, polynucleotide, vector, cell, subunit vaccine, a conjugate vaccine, affinity reagent, vaccine composition or immunotherapy composition or method substantially as herein described.


SEQUENCE LISTING (68 sequences)

SEQ ID NO: 1 - protein, 26 aa, synthetic construct
  REGION 1..26: note = Protein fragment
VYVGKALLTNDTQQEQKLKSQSFTCK

SEQ ID NO: 2 - protein, 24 aa, synthetic construct
  REGION 1..24: note = Protein fragment
THNVPSQDILVPANTTVEVIAYLK

SEQ ID NO: 3 - protein, 47 aa, synthetic construct
  REGION 1..47: note = Protein fragment
DELIVKVRNLNTNNVQEYVIPVDKKEKSNDSNIVKYRSLYIKAPGIK

SEQ ID NO: 4 - protein, 10 aa, synthetic construct
  REGION 1..10: note = Protein fragment mutant
  VARIANT 6: note = Xaa - any amino acid other than Tyr, or is a deletion
RMEKYXPNAM

SEQ ID NO: 5 - protein, 10 aa, synthetic construct
  REGION 1..10: note = Protein fragment mutant
  VARIANT 6: note = Xaa - any amino acid other than Tyr, or is a deletion
GEIPSXLAFP

SEQ ID NO: 6 - protein, 10 aa, synthetic construct
  REGION 1..10: note = Protein fragment mutant
  VARIANT 6: note = Xaa - any amino acid other than His, or is a deletion
SKEITXNVPS

SEQ ID NO: 7 - protein, 10 aa, synthetic construct
  REGION 1..10: note = Protein fragment mutant
  VARIANT 6: note = Xaa - any amino acid other than Val, or is a deletion
LEDVYXGKAL

SEQ ID NO: 8 - protein, 10 aa, synthetic construct
  REGION 1..10: note = Protein fragment mutant
  VARIANT 6: note = Xaa - any amino acid other than Phe, or is a deletion
LKSQSXTCKN

SEQ ID NO: 9 - protein, 10 aa, synthetic construct
  REGION 1..10: note = Protein fragment mutant
  VARIANT 6: note = Xaa - any amino acid other than Val, or is a deletion
NTTVEXIAYL

SEQ ID NO: 10 - protein, 10 aa, synthetic construct
  REGION 1..10: note = Protein fragment mutant
  VARIANT 6: note = Xaa - any amino acid other than Ala, or is a deletion
TVEVIXYLKK

SEQ ID NO: 11 - protein, 328 aa, Clostridium perfringens
MKKNLVKSLAIASAVISIYSIVNIVSPTNVIAKEISNTVSNEMSKKASYDNVDTLIEKGRYNTKYNYLKRMEKYYPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITHNVPSQDILVPANTTVEVIAYLKKVNVKGNVKLVGQVSGSEWGEIPSYLAFPRDGYKFSLSDTVNKSDLNEDGTININGKGNYSAVMGDELIVKVRNLNTNNVQEYVIPVDKKEKSNDSNIVKYRSLSIKAPGIK

SEQ ID NO: 12 - protein, 296 aa, synthetic construct
  REGION 1..296: note = Construct for crystal structure analysis
KEISNTVSNEMSKKASYDNVDTLIEKGRYNTKYNYLKRMEKYYPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITHNVPSQDILVPANTTVEVIAYLKKVNVKGNVKLVGQVSGSEWGEIPSYLAFPRDGYKFSLSDTVNKSDLNEDGTININGKGNYSAVMGDELIVKVRNLNTNNVQEYVIPVDKKEKSNDSNIVKYRSLSIKAPGIK

SEQ ID NO: 13 - protein, 263 aa, Clostridium perfringens
MGKASYDNVDTLIEKGRYNTKYNYLKRMEKYYPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITHNVPSQDILVPANTTVEVIAYLKKVNVKGNVKLVGQVSGSEWGEIPSYLAFPRDGYKFSLSDTVNKSDLNEDGTININGKGNYSAVMGDELIVKVRNLNTNNVQEYVIPVDKK

SEQ ID NO: 14 - protein, 263 aa, synthetic construct
  REGION 1..263: note = Mutant polypeptide
MGKASYDNVDTLIEKGRYNTKYNYLKRMEKYYPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITANVPSQDILVPANTTVEVIAYLKKVNVKGNVKLVGQVSGSEWGEIPSYLAFPRDGYKFSLSDTVNKSDLNEDGTININGKGNYSAVMGDELIVKVRNLNTNNVQEYVIPVDKK

SEQ ID NO: 15 - protein, 315 aa, synthetic construct
  REGION 1..315: note = Recombinant polypeptide
MKYLLPTAAAGLLLLAAQPAMAMGKASYDNVDTLIEKGRYNTKYNYLKRMEKYYPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITHNVPSQDILVPANTTVEVIAYLKKVNVKGNVKLVGQVSGSEWGEIPSYLAFPRDGYKFSLSDTVNKSDLNEDGTININGKGNYSAVMGDELIVKVRNLNTNNVQEYVIPVDKKEKSNDSNIVKYRSLYIKAPGIKLEHHHHHH

SEQ ID NO: 16 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITHNVPSQDILVPANTTVEVIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 17 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITANVPSQDILVPANTTVEVIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 18 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYFGKALLTNDTQQEQKLKSQSFTCKNTDTVTATTTHTVGTSIQATAKFTVPFNETGVSLTTSYSFANTNTNTNSKEITHNVPSQDILVPANTTVEVIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 19 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYVGKALLTNDTQQEQKLKSQSATCKNTDTVTATTTHTVGTSIQATAKFTVPFNETGVSLTTSYSFANTNTNTNSKEITHNVPSQDILVPANTTVEVIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 20 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITHNVPSQDILVPANTTVEAIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 21 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITHNVPSQDILVPANTTVEVIFYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 22 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYFGKALLTNDTQQEQKLKSQSFTCKNTDTVTATTTHTVGTSIQATAKFTVPFNETGVSLTTSYSFANTNTNTNSKEITANVPSQDILVPANTTVEVIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 23 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYVGKALLTNDTQQEQKLKSQSATCKNTDTVTATTTHTVGTSIQATAKFTVPFNETGVSLTTSYSFANTNTNTNSKEITANVPSQDILVPANTTVEVIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 24 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITANVPSQDILVPANTTVEAIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 25 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITANVPSQDILVPANTTVEVIFYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 26 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYFGKALLTNDTQQEQKLKSQSATCKNTDTVTATTTHTVGTSIQATAKFTVPFNETGVSLTTSYSFANTNTNTNSKEITHNVPSQDILVPANTTVEVIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 27 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYFGKALLTNDTQQEQKLKSQSFTCKNTDTVTATTTHTVGTSIQATAKFTVPFNETGVSLTTSYSFANTNTNTNSKEITHNVPSQDILVPANTTVEAIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 28 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYFGKALLTNDTQQEQKLKSQSFTCKNTDTVTATTTHTVGTSIQATAKFTVPFNETGVSLTTSYSFANTNTNTNSKEITHNVPSQDILVPANTTVEVIFYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 29 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYVGKALLTNDTQQEQKLKSQSATCKNTDTVTATTTHTVGTSIQATAKFTVPFNETGVSLTTSYSFANTNTNTNSKEITHNVPSQDILVPANTTVEAIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 30 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYVGKALLTNDTQQEQKLKSQSATCKNTDTVTATTTHTVGTSIQATAKFTVPFNETGVSLTTSYSFANTNTNTNSKEITHNVPSQDILVPANTTVEVIFYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 31 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITHNVPSQDILVPANTTVEAIFYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 32 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYFGKALLTNDTQQEQKLKSQSATCKNTDTVTATTTHTVGTSIQATAKFTVPFNETGVSLTTSYSFANTNTNTNSKEITANVPSQDILVPANTTVEVIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 33 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYFGKALLTNDTQQEQKLKSQSFTCKNTDTVTATTTHTVGTSIQATAKFTVPFNETGVSLTTSYSFANTNTNTNSKEITANVPSQDILVPANTTVEAIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 34 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYFGKALLTNDTQQEQKLKSQSFTCKNTDTVTATTTHTVGTSIQATAKFTVPFNETGVSLTTSYSFANTNTNTNSKEITANVPSQDILVPANTTVEVIFYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 35 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYVGKALLTNDTQQEQKLKSQSATCKNTDTVTATTTHTVGTSIQATAKFTVPFNETGVSLTTSYSFANTNTNTNSKEITANVPSQDILVPANTTVEAIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 36 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYVGKALLTNDTQQEQKLKSQSATCKNTDTVTATTTHTVGTSIQATAKFTVPFNETGVSLTTSYSFANTNTNTNSKEITANVPSQDILVPANTTVEVIFYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 37 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITANVPSQDILVPANTTVEAIFYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 38 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYFGKALLTNDTQQEQKLKSQSATCKNTDTVTATTTHTVGTSIQATAKFTVPFNETGVSLTTSYSFANTNTNTNSKEITHNVPSQDILVPANTTVEAIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 39 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYFGKALLTNDTQQEQKLKSQSATCKNTDTVTATTTHTVGTSIQATAKFTVPFNETGVSLTTSYSFANTNTNTNSKEITHNVPSQDILVPANTTVEVIFYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 40 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYFGKALLTNDTQQEQKLKSQSFTCKNTDTVTATTTHTVGTSIQATAKFTVPFNETGVSLTTSYSFANTNTNTNSKEITHNVPSQDILVPANTTVEAIFYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 41 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYVGKALLTNDTQQEQKLKSQSATCKNTDTVTATTTHTVGTSIQATAKFTVPFNETGVSLTTSYSFANTNTNTNSKEITHNVPSQDILVPANTTVEAIFYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 42 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYFGKALLTNDTQQEQKLKSQSATCKNTDTVTATTTHTVGTSIQATAKFTVPFNETGVSLTTSYSFANTNTNTNSKEITANVPSQDILVPANTTVEAIAYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 43 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYFGKALLTNDTQQEQKLKSQSATCKNTDTVTATTTHTVGTSIQATAKFTVPFNETGVSLTTSYSFANTNTNTNSKEITANVPSQDILVPANTTVEVIFYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 44 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYFGKALLTNDTQQEQKLKSQSATCKNTDTVTATTTHTVGTSIQATAKFTVPFNETGVSLTTSYSFANTNTNTNSKEITHNVPSQDILVPANTTVEAIFYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 45 - protein, 263 aa, synthetic construct
  REGION 1..263: note = Mutant polypeptide
MGKASYDNVDTLIEKGRYNTKYNYLKRMEKYAPNAMAYFDKVTINPQGNDFYINNPKVELDGEPSMNYLEDVYFGKALLTNDTQQEQKLKSQSATCKNTDTVTATTTHTVGTSIQATAKFTVPFNETGVSLTTSYSFANTNTNTNSKEITANVPSQDILVPANTTVEAIFYLKKVNVKGNVKLVGQVSGSEWGEIPSALAFPRDGYKFSLSDTVNKSDLNEDGTININGKGNYSAVMGDELIVKVRNLNTNNVQEYVIPVDKK

SEQ ID NO: 46 - protein, 263 aa, synthetic construct
  REGION 1..263: note = Mutant polypeptide
MGKASYDNVDTLIEKGRYNTKYNYLKRMEKYYPNAMAYFDKVTINPQGNDFYINNPKVELDGEPSMNYLEDVYFGKALLTNDTQQEQKLKSQSFTCKNTDTVTATTTHTVGTSIQATAKFTVPFNETGVSLTTSYSFANTNTNTNSKEITHNVPSQDILVPANTTVEVIAYLKKVNVKGNVKLVGQVSGSEWGEIPSYLAFPRDGYKFSLSDTVNKSDLNEDGTININGKGNYSAVMGDELIVKVRNLNTNNVQEYVIPVDKK

SEQ ID NO: 47 - protein, 263 aa, synthetic construct
  REGION 1..263: note = Mutant polypeptide
MGKASYDNVDTLIEKGRYNTKYNYLKRMEKYYPNAMAYFDKVTINPQGNDFYINNPKVELDGEPSMNYLEDVYVGKALLTNDTQQEQKLKSQSATCKNTDTVTATTTHTVGTSIQATAKFTVPFNETGVSLTTSYSFANTNTNTNSKEITHNVPSQDILVPANTTVEVIAYLKKVNVKGNVKLVGQVSGSEWGEIPSYLAFPRDGYKFSLSDTVNKSDLNEDGTININGKGNYSAVMGDELIVKVRNLNTNNVQEYVIPVDKK

SEQ ID NO: 48 - protein, 263 aa, synthetic construct; identical to SEQ ID NO: 14
  REGION 1..263: note = Mutant polypeptide

SEQ ID NO: 49 - protein, 263 aa, synthetic construct
  REGION 1..263: note = Mutant polypeptide
MGKASYDNVDTLIEKGRYNTKYNYLKRMEKYYPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITHNVPSQDILVPANTTVEAIAYLKKVNVKGNVKLVGQVSGSEWGEIPSYLAFPRDGYKFSLSDTVNKSDLNEDGTININGKGNYSAVMGDELIVKVRNLNTNNVQEYVIPVDKK

SEQ ID NO: 50 - protein, 263 aa, synthetic construct
  REGION 1..263: note = Mutant polypeptide
MGKASYDNVDTLIEKGRYNTKYNYLKRMEKYYPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITHNVPSQDILVPANTTVEVIFYLKKVNVKGNVKLVGQVSGSEWGEIPSYLAFPRDGYKFSLSDTVNKSDLNEDGTININGKGNYSAVMGDELIVKVRNLNTNNVQEYVIPVDKK

SEQ ID NO: 51 - DNA, 83 bp, synthetic construct
  misc_feature 1..83: note = Primer sequence
  source 1..83: mol_type = other DNA
gaaaggaagatataatacaaaatataattacttaaagagaatggaaaaatatgcgcctaatgctatggcatattttgataagg

SEQ ID NO: 52 - DNA, 83 bp, synthetic construct
  misc_feature 1..83: note = Primer sequence
  source 1..83: mol_type = other DNA
ccttatcaaaatatgccatagcattaggcgcatatttttccattctctttaagtaattatattttgtattatatcttcctttc

SEQ ID NO: 53 - DNA, 49 bp, synthetic construct
  misc_feature 1..49: note = Primer sequence
  source 1..49: mol_type = other DNA
gtgaatggggagagatacctagtgcgttagcttttcctagggatggtta

SEQ ID NO: 54 - DNA, 49 bp, synthetic construct
  misc_feature 1..49: note = Primer sequence
  source 1..49: mol_type = other DNA
taaccatccctaggaaaagctaacgcactaggtatctctccccattcac

SEQ ID NO: 55 - DNA, 65 bp, synthetic construct
  misc_feature 1..65: note = Primer sequence
  source 1..65: mol_type = other DNA
caaatacaaatacaaatactaattcaaaagaaattactgctaatgtcccttcacaagatatacta

SEQ ID NO: 56 - DNA, 65 bp, synthetic construct
  misc_feature 1..65: note = Primer sequence
  source 1..65: mol_type = other DNA
tagtatatcttgtgaagggacattagcagtaatttcttttgaattagtatttgtatttgtatttg

SEQ ID NO: 57 - DNA, 54 bp, synthetic construct
  misc_feature 1..54: note = Primer sequence
  source 1..54: mol_type = other DNA
agaaccatcaatgaattatcttgaagatgtttattttggaaaagctctcttaac

SEQ ID NO: 58 - DNA, 54 bp, synthetic construct
  misc_feature 1..54: note = Primer sequence
  source 1..54: mol_type = other DNA
gttaagagagcttttccaaaataaacatcttcaagataattcattgatggttct

SEQ ID NO: 59 - DNA, 76 bp, synthetic construct
  misc_feature 1..76: note = Primer sequence
  source 1..76: mol_type = other DNA
tcttaactaatgatactcaacaagaacaaaaattaaaatcacaatcagcgacttgtaaaaatactgatacagtaac

SEQ ID NO: 60 - DNA, 76 bp, synthetic construct
  misc_feature 1..76: note = Primer sequence
  source 1..76: mol_type = other DNA
gttactgtatcagtatttttacaagtcgctgattgtgattttaatttttgttcttgttgagtatcattagttaaga

SEQ ID NO: 61 - DNA, 64 bp, synthetic construct
  misc_feature 1..64: note = Primer sequence
  source 1..64: mol_type = other DNA
atactagtaccagctaatactactgtagaagcgatagcatatttaaaaaaagttaatgttaaag

SEQ ID NO: 62 - DNA, 64 bp, synthetic construct
  misc_feature 1..64: note = Primer sequence
  source 1..64: mol_type = other DNA
ctttaacattaactttttttaaatatgctatcgcttctacagtagtattagctggtactagtat

SEQ ID NO: 63 - DNA, 82 bp, synthetic construct
  misc_feature 1..82: note = Primer sequence
  source 1..82: mol_type = other DNA
gatatactagtaccagctaatactactgtagaagtaatattttatttaaaaaaagttaatgttaaaggaaatgtaaagttag

SEQ ID NO: 64 - DNA, 82 bp, synthetic construct
  misc_feature 1..82: note = Primer sequence
  source 1..82: mol_type = other DNA
ctaactttacatttcctttaacattaactttttttaaataaaatattacttctacagtagtattagctggtactagtatatc

SEQ ID NO: 65 - protein, 260 aa, synthetic construct
  REGION 1..260: note = Protein fragment
KASYDNVDTLIEKGRYNTKYNYLKRMEKYYPNAMAYFDKVTINPQGNDFYINNPKVELDGEPSMNYLEDVYVGKALLTNDTQQEQKLKSQSFTCKNTDTVTATTTHTVGTSIQATAKFTVPFNETGVSLTTSYSFANTNTNTNSKEITHNVPSQDILVPANTTVEVIAYLKKVNVKGNVKLVGQVSGSEWGEIPSYLAFPRDGYKFSLSDTVNKSDLNEDGTININGKGNYSAVMGDELIVKVRNLNTNNVQEYVIPVDK

SEQ ID NO: 66 - protein, 32 aa, synthetic construct
  REGION 1..32: note = Protein fragment
MKKNLVKSLAIASAVISIYSIVNIVSPTNVIA

SEQ ID NO: 67 - protein, 13 aa, synthetic construct
  REGION 1..13: note = Protein fragment
KEISNTVSNEMSK

SEQ ID NO: 68 - protein, 23 aa, synthetic construct
  REGION 1..23: note = Protein fragment
KEKSNDSNIVKYRSLYIKAPGIK